SEQUENCE LISTING (17 sequences)

SEQ ID NO: 1 - DNA, 4242 bp, Artificial sequence
  Nucleotide sequence of plasmid pGCMB75.6
tgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagccgatctgataacggtaccgataagctggcggccgattaagctacagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccaggttcaattacagctcttaagcagccgcaagcttgatatcgaattcctgcagcccgggggatccactagttctagagcggccgccaccgcggtggagctcgaattatcagatcgattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcatcagatctgccggtctccctatagtgagtcgtattaatttcgataagccaggttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgggcgcgcctaggcttttgcaaagatcgatcaagagacaggatgaggatcgtttcgcagcttttcattctgactgcaacgggcaataagtctctgtgtggattaaaaaaagagtgtctgatagcagcttctgaactggttacctgccgtgagtaaattaaaattttattgacttaggtcactaaggcgccttgcgctgaggttgcgtcgtgatatcatcagggcagaccggttacatccccctaacaagctgtataaagagaaatactatctcattggcgttgcccgcacctgacagtgcgacgttgggctgcgtccgtcgaccaacggtaccgaggtaacagcccaatctatccatgatctcggccaggccgggtcggccgttatgcagcccggctcgggtatgaagccattaaggagccgacccagcgcgaccgggcggccggtcacgctgcctctgctgaagcctgcctgtcactccctgcgcggcgtacccgccgttctcatcgagtaggctccggatcgcgaccccggacgggccctgggcccaggagcggcctatgacaaatgccgggtagcgatccggcattcagcattgactgcgcacggatccagtccttgcaggagccttatgccgaccgtagcaaaaaatgagcccgagccgatcgcgagttgtgatccggtcccgccgattgccggtcgcgatgacggtcctgtgtaagcgttatcgttaccaattgtttaagaagtatatacgctacgaggtacttgataacttctgcgtagcatacatgaggttttgtataaaaatggcgggcgatatcaacgcagtgtcagaaatccgaaacagtctgcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctaggcgcgctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgagcaggatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggc

SEQ ID NO: 2 - DNA, 4242 bp, Artificial sequence
  Nucleotide sequence of plasmid pMB75.6
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgagcaggatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagccgatctgataacggtaccgataagctggcggccgattaagctacagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccaggttcaattacagctcttaagcagccgcaagcttgatatcgaattcctgcagcccgggggatccactagttctagagcggccgccaccgcggtggagctcgaattatcagatcgattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcatcagatctgccggtctccctatagtgagtcgtattaatttcgataagccaggttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagcgcgcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctaggcgcgctcatgagcggatacatatttgaatgtatttagaaaaataaacaaataggggttccgcgcacatttccccgaaaagtgccac

SEQ ID NO: 3 - DNA, 4242 bp, Artificial sequence
  Nucleotide sequence of plasmid pMB75.6_AscI
tgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagccgatctgataacggtaccgataagctggcggccgattaagctacagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccaggttcaattacagctcttaagcagccgcaagcttgatatcgaattcctgcagcccgggggatccactagttctagagcggccgccaccgcggtggagctcgaattatcagatcgattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcatcagatctgccggtctccctatagtgagtcgtattaatttcgataagccaggttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgggcgcgcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctaggcgcgctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgagcaggatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggc

SEQ ID NO: 4 - DNA, 4242 bp, Artificial sequence
  Nucelotide sequence of plasmid pLacZMB75.6
tgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagccgatctgataacggtaccgataagctggcggccgattaagctacagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccaggttcaattacagctcttaagcagccgccaaaacaaaattcctcaaaaatcatcatcgaatgaatggtgaaataatttccctgaataactgtagtgttttcagggcgcggcataataattaactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcatcagatctgccggtctccctatagtgagtcgtattaatttcgataagccaggttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgggcgcgcctaggcttttgcaaagatcgatcaagagacaggatgaggatcgtttcgcagcttttcattctgactgcaacgggcaataagtctctgtgtggattaaaaaaagagtgtctgatagcagcttctgaactggttacctgccgtgagtaaattaaaattttattgacttaggtcactaaggcgccttgcgctgaggttgcgtcgtgatatcatcagggcagaccggttacatccccctaacaagctgtataaagagaaatactatctcattggcgttgcccgcacctgacagtgcgacgttgggctgcgtccgtcgaccaacggtaccgaggtaacagcccaatctatccatgatctcggccaggccgggtcggccgttatgcagcccggctcgggtatgaagccattaaggagccgacccagcgcgaccgggcggccggtcacgctgcctctgctgaagcctgcctgtcactccctgcgcggcgtacccgccgttctcatcgagtaggctccggatcgcgaccccggacgggccctgggcccaggagcggcctatgacaaatgccgggtagcgatccggcattcagcattgactgcgcacggatccagtccttgcaggagccttatgccgaccgtagcaaaaaatgagcccgagccgatcgcgagttgtgatccggtcccgccgattgccggtcgcgatgacggtcctgtgtaagcgttatcgttaccaattgtttaagaagtatatacgctacgaggtacttgataacttctgcgtagcatacatgaggttttgtataaaaatggcgggcgatatcaacgcagtgtcagaaatccgaaacagtctgcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctaggcgcgctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgatatcgaattcctgcagcccgggggatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgc

SEQ ID NO: 5 - DNA, 20 bp, Artificial sequence
  Primer
gtgcgcggaacccctatttg

SEQ ID NO: 6 - DNA, 20 bp, Artificial sequence
  Primer
gcgtacccgccgttctcatc

SEQ ID NO: 7 - DNA, 20 bp, Artificial sequence
  Primer
gtctgacgctcagtggaacg

SEQ ID NO: 8 - DNA, 22 bp, Artificial sequence
  Primer
gttccagtttggaacaagagtc

SEQ ID NO: 9 - DNA, 20 bp, Artificial sequence
  Primer
ggcaattagccatattagtc

SEQ ID NO: 10 - DNA, 22 bp, Artificial sequence
  Primer
gcagagctcgtttagtgaaccg

SEQ ID NO: 11 - DNA, 21 bp, Artificial sequence
  Primer
gatcataatcagccataccac

SEQ ID NO: 12 - DNA, 27 bp, Artificial sequence
  Primer
cgtcttgagtccaacccggtaagacac

SEQ ID NO: 13 - DNA, 24 bp, Artificial sequence
  Primer
ccacaggtgtccactcccaggttc

SEQ ID NO: 14 - DNA, 21 bp, Artificial sequence
  Primer
ctagtcaaggcactatacatc

SEQ ID NO: 15 - DNA, 22 bp, Artificial sequence
  Primer
tccacagaatcaggggataacg

SEQ ID NO: 16 - DNA, 23 bp, Artificial sequence
  Primer
gctcactcattaggcaccccagg

SEQ ID NO: 17 - DNA, 22 bp, Artificial sequence
  Primer
cgcgtaatacgactcactatag

(19) United States
(12) Patent Application Publication
(10) Pub. No.: US 2019/0008952 A1
(43) Pub. Date: Jan. 10, 2019

(54) ENHANCED IMMUNE RESPONSE IN AQUATIC SPECIES

(71) Applicant: BAYER ANIMAL HEALTH GMBH, Leverkusen (DE)

(72) Inventors: Christian WEISS, Leverkusen (DE); Nathalie Simard, Penicuik (GB); Jan Koesling, Berlin (DE)

(21) Appl. No.: 15/748,761

(22) PCT Filed: Jul. 27, 2016
(86) PCT No.: PCT/EP2016/067850

§ 371 Date: Jan. 30, 2018

Related U.S. Application Data
(60) Provisional application No. 62/199,840, filed on Jul. 31, 2015.

(51) Int. Cl. A61K 39/39; A61K 39/12

(52) U.S. Cl. CPC A61K 39/39; A61K 2039/5252; A61K 39/12

(57) ABSTRACT

The present invention generally relates to methods of eliciting an immune response in an aquatic species subject. In particular, an immunomodulator composition is used to induce an immune response to enhance the subject's ability to fight pathogens.

### CROSS-REFERENCE TO RELATED APPLICATIONS

This application claims the benefit of PCT International Application No. PCT/EP2016/067850, filed Jul. 27, 2016, which claims the benefit of U.S. Patent Application Ser. No. 62/199,840, filed Jul. 31, 2015, the contents of which are herein incorporated by reference in their entirety.

### FIELD OF THE INVENTION

The present invention generally relates to methods of eliciting an immune response in a subject by activating innate immunity. In particular, an immunomodulator composition is used to stimulate innate immunity in a member of an aquatic species.

### SUMMARY OF THE INVENTION

The present invention relates to methods of using immunostimulatory plasmids to modulate innate immunity in an aquatic species subject. The immunostimulatory plasmid may comprise a nucleic acid sequence having at least 80% sequence identity with the sequence of SEQ ID NO: 1, SEQ ID NO: 2, SEQ ID NO: 3, SEQ ID NO: 4, or a combination thereof. In some aspects, the immunostimulatory plasmid may comprise a nucleic acid molecule having at least 84% sequence identity with the sequence of SEQ ID NO: 4. In some aspects, the immunostimulatory plasmid may comprise the sequence of SEQ ID NO: 1. In some aspects, the immunostimulatory plasmid may comprise the sequence of SEQ ID NO: 4. In some aspects, the immunostimulatory plasmid may comprise the sequence of SEQ ID NO: 2. In some aspects, the immunostimulatory plasmid may comprise the sequence of SEQ ID NO: 3.

In other aspects, the immunostimulatory plasmid may consist of a nucleic acid sequence having at least 80% sequence identity with the sequence of SEQ ID NO: 1, SEQ ID NO: 2, SEQ ID NO: 3, SEQ ID NO: 4 or a combination thereof. In some aspects, the immunostimulatory plasmid may consist of a nucleic acid molecule having at least 84% sequence identity with the sequence of SEQ ID NO: 4. In some aspects, the immunostimulatory plasmid may consist of the sequence of SEQ ID NO: 1. In some aspects, the immunostimulatory plasmid may consist of the sequence of SEQ ID NO: 4. In some aspects, the immunostimulatory plasmid may consist of the sequence of SEQ ID NO: 2. In some aspects, the immunostimulatory plasmid may consist of the sequence of SEQ ID NO: 3.

In some aspects, the immunostimulatory plasmid preferably does not comprise a nucleic acid sequence encoding a full-length or functional selectable or screenable marker. In other aspects, the immunostimulatory plasmid comprises a nucleic acid sequence encoding a selectable or screenable marker that is not an antibiotic resistance gene.

The present invention also relates to pharmaceutical formulations comprising any of the immunostimulatory plasmids, or DNA sequences, described herein and a pharmaceutically acceptable carrier.

The present invention further relates to immunomodulator compositions comprising a cationic liposome delivery vehicle and any of the immunostimulatory plasmids, or DNA sequences, described herein.

In some aspects, the present invention relates to methods of using the immunostimulatory plasmids, or DNA sequences, described herein. Suitable methods of use include therapeutic administration to a subject of the aquatic species. Such therapeutic administration includes prophylactic treatment, metaphylactic treatment, and post-infection treatment of a subject or subjects.

The present invention relates to methods of stimulating or eliciting an immune response in a subject. In some aspects, the methods include stimulating an immune response in a subject by administering to the subject an immunomodulator composition described herein. In some aspects, the methods include stimulating an immune response in a subject by administering to the subject an immunostimulatory plasmid, or DNA sequence, described herein.

Other objects and features will be in part apparent and in part pointed out hereinafter.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows a map of the pMB75.6 plasmid (SEQ ID NO: 2);

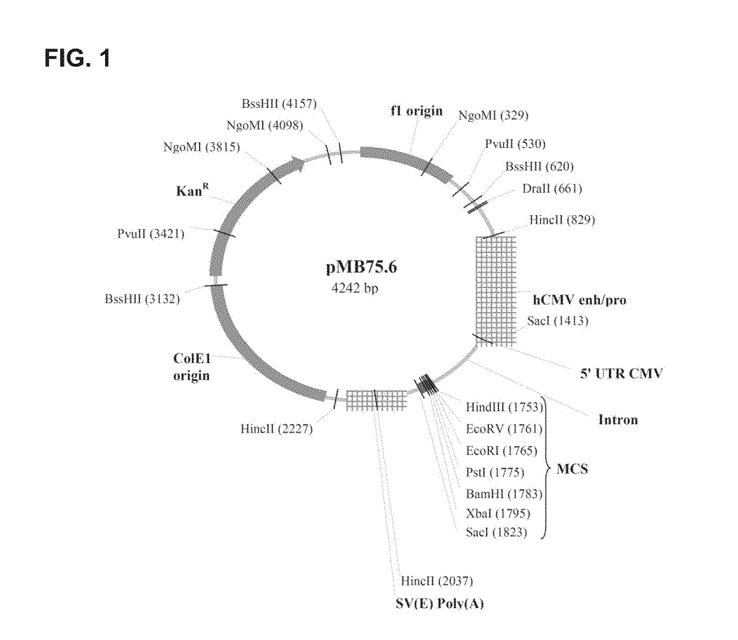

FIG. 2 shows a map of the pGCMB75.6 plasmid (SEQ ID NO: 1);

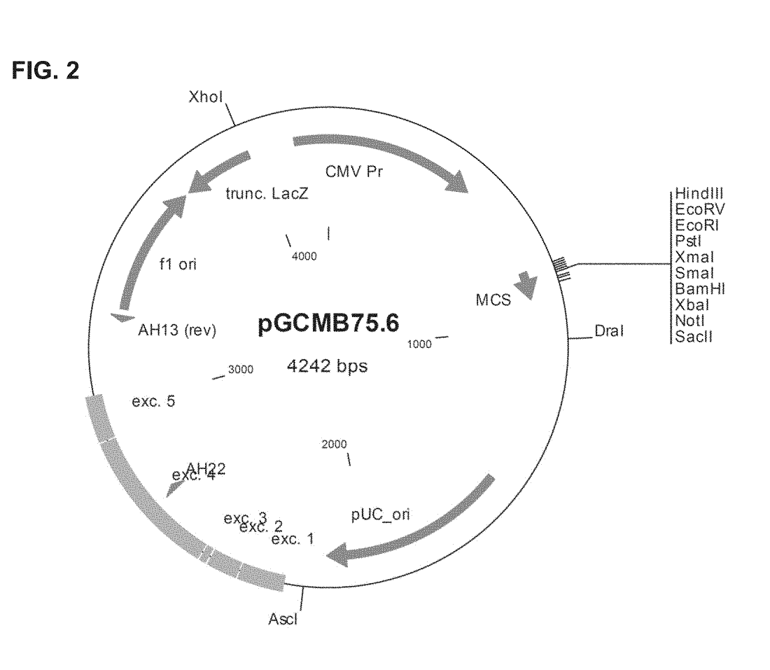

FIG. 3 shows a map of the pLacZ75.6 plasmid (SEQ ID NO: 4);

FIG. 4 graphically illustrates the daily mortality percentage post-challenge for fish tank M5.

FIG. 5 graphically illustrates the daily mortality percentage post-challenge for fish tank M6.

FIG. 6 shows Kaplan-Meier survival curves for combined mortalities from each group of the vaccine study. Vertical axis is the probability of the survival at a specific time DPC (horizontal axis). Crosses at the end of the curve represent censored data (fish alive beyond 27 DPC). DPC: days post challenge.

FIG. 7 depicts a pair-wise comparison between the 12 groups of fish from the vaccine study, with linked groups (represented as nodes displayed by the green circles) showing no significant difference between them (α=0.05).

FIG. 8 graphically illustrates the relative percentage of survival for vaccine study groups when compared with the dextrose 5%-treated control group. Comparisons were made between the cumulative mortality at the end of experiment (RPSend). Significant cumulative mortalities differences (α=0.05) between the dextrose 5% group and the other treatments are indicated with *.

### DETAILED DESCRIPTION OF THE INVENTION

In accordance with the present invention, a composition capable of eliciting an immune response in a recipient subject, as well as methods of use, have been discovered. In particular, the present invention relates to nucleic acid compositions, or immunomodulator compositions, and uses thereof. It has been discovered that such immunomodulator compositions may be used to modulate the immune system of a member of aquatic species. The invention is particularly useful in the treatment and prevention of infectious diseases caused by microorganisms, such as, without limitation, viruses, bacteria, mold, fungus, yeast, parasites and other microbes known in the art. The compositions and methods of using the immunomodulator compositions are discussed in more detail below.

I. Compositions

Compositions useful in this invention, such as those described herein, are generally able to be used as a prophylactic therapy, metaphylactic therapy, or treatment therapy for infectious diseases. Such compositions are referred to herein as immunomodulator compositions. The immunomodulator compositions include at least an immunostimulatory plasmid, or immunostimulatory DNA sequence, capable of inducing an immune response in a recipient subject. In some aspects, the immune response is an innate immune response. In some aspects, the immune response is a combination of innate immune response and acquired immune response. In some aspects, the immunomodulator compositions may also include a liposome delivery vehicle.

A. Nucleic Acids

In some aspects the present invention relates to nucleic acid molecules useful for the treatment or prevention of infectious disease causing agents. The nucleic acid molecules described herein may be included in an immunostimulatory plasmid, as linear double stranded or single stranded DNA, amino acid sequence, ribonucleic acid (RNA), or combinations thereof. In some aspects, the present invention relates to nucleic acid molecules, vectors, and host cells (in vitro, in vivo, or ex vivo) which contain the immunostimulatory plasmid or immunostimulatory DNA sequence.

The nucleic acid molecules described herein are enriched in CpG motifs. Such CpG motifs may induce immune stimulation via immune surveillance receptors including specific Toll-like receptors, such as TLR9 and TLR21. In addition the nucleic acid molecules described herein also contain non-CpG immunostimulatory motifs. In some aspects, the nucleic acid molecules contain about 2-20% CpG motifs over the frequency of CpG motifs expected in random nucleic acid sequences. In some aspects, the nucleic acid molecules contain about 3, 4, 5, 6, 7, 8, 9, 10, 11, 12, 13, 14, 15, 16, 17, 18, 19, 20, 25, 30, 35, 40%, or more CpG motifs over the frequency of CpG motifs expected in random nucleic acid sequences. In some aspects, the nucleic acid molecules contain about 10% CpG motifs over the frequency of CpG motifs expected in random nucleic acid sequences. In some aspects, compared to vertebrate DNA, an enrichment of CpG motifs of more than 10-fold is observed. In some aspects, the nucleic acid molecules contain about 2 to 50 fold, or more CpG motifs compared to vertebrate DNA. In some aspects, the nucleic acid molecules contain about 3, 4, 5, 6, 7, 8, 9, 10, 11, 12, 13, 14, 15, 16, 17, 18, 19, 20, 25, 30, 35, 40, 45, 50, 55 fold or more CpG motifs compared to vertebrate DNA.

In some aspects, the present invention relates to immunostimulatory plasmids, or DNA sequences, that do not comprise an antibiotic resistance gene. The plasmids may be devoid of any selectable or screenable marker genes. For example, the pGCMB75.6 plasmid described herein does not comprise any full-length or functional selectable or screenable marker genes. The sequence of pGCMB75.6 is provided in SEQ ID NO: 1.

In some aspects, the immunostimulatory plasmids described herein preferably do not comprise a nucleic acid sequence coding for a full-length or functional selectable or screenable marker. In some aspects, the immunostimulatory plasmids do not comprise an antibiotic resistance gene. For example, the plasmids do not comprise a kanamycin resistance gene. In some aspects, the plasmids described herein preferably do not encode an immunogen.

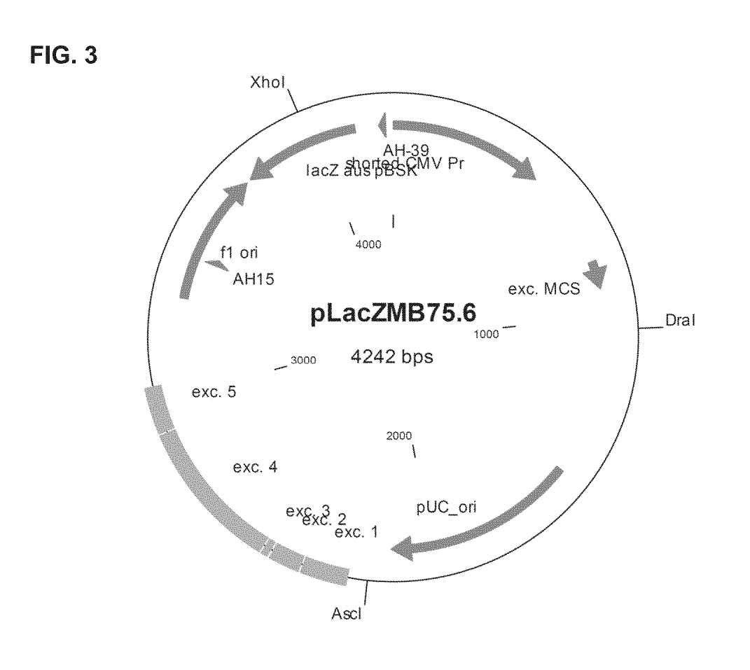

In some aspects, the immunostimulatory plasmids may comprise a nucleic acid sequence coding for a selectable or screenable marker gene that is not an antibiotic resistance gene. For example, the pLacZMB75.6 plasmid described herein comprises a LacZ gene as a screenable marker. A map of pLacZMB75.6 is provided in FIG. 3 and the nucleotide sequence of pLacZMB75.6 is provided as SEQ ID NO: 4. As shown in FIG. 3, pLacZMB75.6 is similar to pGCMB75.6, but contains a LacZ screenable marker.

It will be appreciated that the nucleotide sequences of the pMB75.6 (SEQ ID NO: 2 OR SEQ ID NO: 3 (p MB75.6 with an AscI restriction site)), pGCMB75.6 (SEQ ID NO: 1) or pLacZMB75.6 (SEQ ID NO: 4) plasmids may be varied to a certain extent without significantly adversely affecting their immunostimulatory properties. In some aspects, the present invention relates to an immunostimulatory plasmid comprising a nucleic acid sequence having at least 89% sequence identity with the sequence of pGCMB75.6 (SEQ ID NO: 1). The immunostimulatory plasmid preferably comprises a nucleic acid sequence having at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity with the sequence of pGCMB75.6 (SEQ ID NO: 1). In some aspects, the immunostimulatory plasmid more preferably comprises the sequence of pGCMB75.6 (SEQ ID NO: 1).

In some aspects, the present invention relates to an immunostimulatory plasmid comprising a nucleic acid sequence having at least 84% sequence identity with the sequence of pLacZMB75.6 (SEQ ID NO: 4). The immunostimulatory plasmid preferably comprises a nucleic acid sequence having at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity with the sequence of pLacZMB75.6 (SEQ ID NO: 4). In some aspects, the immunostimulatory plasmid more preferably comprises the sequence of pLacZMB75.6 (SEQ ID NO: 4).

In some aspects, the present invention relates to an immunostimulatory plasmid comprising a nucleic acid sequence having at least 80% sequence identity with the sequence of SEQ ID NO: 2. The immunostimulatory plasmid preferably comprises a nucleic acid sequence having at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity with the sequence of SEQ ID NO: 2. In some aspects, the immunostimulatory plasmid more preferably comprises the sequence of SEQ ID NO: 2.

In some aspects, the present invention relates to an immunostimulatory plasmid comprising a nucleic acid sequence having at least 80% sequence identity with the sequence of SEQ ID NO: 3. The immunostimulatory plasmid preferably comprises a nucleic acid sequence having at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity with the sequence of SEQ ID NO: 3. In some aspects, the immunostimulatory plasmid more preferably comprises the sequence of SEQ ID NO: 3.

In some aspects, the present invention relates to an immunostimulatory plasmid consisting of a nucleic acid sequence having at least 89% sequence identity with the sequence of pGCMB75.6 (SEQ ID NO: 1). The immunostimulatory plasmid preferably consists of a nucleic acid sequence having at least 75%, at least 76%, at least 77%, at least 78%, at least 79%, at least 80%, at least 81%, at least 82%, at least 83%, at least 84%, at least, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity with the sequence of pGCMB75.6 (SEQ ID NO: 1). In some aspects, the immunostimulatory plasmid more preferably consists of the sequence of pGCMB75.6 (SEQ ID NO: 1).

In some aspects, the present invention relates to an immunostimulatory plasmid consisting of a nucleic acid sequence having at least 84% sequence identity with the sequence of pLacZMB75.6 (SEQ ID NO: 4). The immunostimulatory plasmid preferably consists of a nucleic acid sequence having at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity with the sequence of pLacZMB75.6 (SEQ ID NO: 4). In some aspects, the immunostimulatory plasmid more preferably consists of the sequence of pLacZMB75.6 (SEQ ID NO: 4).

In some aspects, the present invention relates to an immunostimulatory plasmid consisting of a nucleic acid sequence having at least 80% sequence identity with the sequence of SEQ ID NO: 2. The immunostimulatory plasmid preferably consists of a nucleic acid sequence having at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity with the sequence of SEQ ID NO: 2. In some aspects, the immunostimulatory plasmid more preferably consists of the sequence of SEQ ID NO: 2.

In some aspects, the present invention relates to an immunostimulatory plasmid consisting of a nucleic acid sequence having at least 80% sequence identity with the sequence of SEQ ID NO: 3. The immunostimulatory plasmid preferably consists of a nucleic acid sequence having at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity with the sequence of SEQ ID NO: 3. In some aspects, the immunostimulatory plasmid more preferably consists of the sequence of SEQ ID NO: 3.

Another important aspect of this invention provides for immunostimulatory DNA sequences or immunostimulatory plasmids capable of stimulating an immune response including nucleic acid sequences that hybridize under high stringency conditions to SEQ ID NO: 1, SEQ ID NO: 2, SEQ ID NO: 3, or SEQ ID NO: 4. Suitable nucleic acid sequences include those that are homologous, substantially similar, or identical to the nucleic acids of the present invention. In some aspects, homologous nucleic acid sequences will have a sequence similarity of at least about 75%, 76%, 77%, 78%, 79%, 80% 81%, 82%, 83%, 84%, 85%, 86%, 87%, 88%, 89%, 90%, 91%, 92%, 93%, 94%, 95%, 96%, 97%, 98%, 99%, or 100% to SEQ ID NO: 1 or the respective complementary sequence. In other aspects, homologous nucleic acid sequences will have a sequence similarity of at least about 75%, 76%, 77%, 78%, 79%, 80% 81%, 82%, 83%, 84%, 85%, 86%, 88%, 89%, 90%, 91%, 92%, 93%, 94%, 95%, 96%, 97%, 98%, 99%, or 100% to SEQ ID NO: 4 or the respective complementary sequence. In other aspects, homologous nucleic acid sequences will have a sequence similarity of at least about 75%, 76%, 77%, 78%, 79%, 80% 81%, 82%, 83%, 84%, 85%, 86%, 88%, 89%, 90%, 91%, 92%, 93%, 94%, 95%, 96%, 97%, 98%, 99%, or 100% to SEQ ID NO: 2 or the respective complementary sequence. In other aspects, homologous nucleic acid sequences will have a sequence similarity of at least about 75%, 76%, 77%, 78%, 79%, 80% 81%, 82%, 83%, 84%, 85%, 86%, 88%, 89%, 90%, 91%, 92%, 93%, 94%, 95%, 96%, 97%, 98%, 99%, or 100% to SEQ ID NO: 3 or the respective complementary sequence. Sequence similarity may be calculated using a number of algorithms known in the art, such as BLAST, described in Altschul, S. F., et al., J. Mol. Biol. 215:403-10, 1990. The nucleic acids may differ in sequence from the above-described nucleic acids due to the degeneracy of the genetic code. In general, a reference sequence will be 18 nucleotides, more usually 30 or more nucleotides, and may comprise the entire nucleic acid sequence of the composition for comparison purposes.

Nucleotide sequences that can hybridize to SEQ ID NO: 1, SEQ ID NO: 2, SEQ ID NO: 3, or SEQ ID NO: 4 are contemplated herein. Stringent hybridization conditions include conditions such as hybridization at 50° C. or higher and 0.1×SSC (15 mM sodium chloride/1.5 mM sodium citrate). Another example is overnight incubation at 42° C. in a solution of 50% formamide, 5×SSC (150 mM NaCl, 15 mM trisodium citrate), 50 mM sodium phosphate (pH 7.6), 5×Denhardt's solution, 10% dextran sulfate, and 20 μg/ml denatured, sheared salmon sperm DNA, followed by washing in 0.1×SSC at about 65° C. Exemplary stringent hybridization conditions are hybridization conditions that are at least about 80%, 85%, 90%, or 95% as stringent as the above specific conditions. Other stringent hybridization conditions are known in the art and may also be employed to identify homologs of the nucleic acids of the invention (Current Protocols in Molecular Biology, Unit 6, pub. John Wiley & Sons, N.Y. 1989).

Mutant nucleotides of the DNA molecules described herein may be used, so long as mutants include nucleic acid sequences maintain the ability to stimulate an innate immune response as described herein. The DNA sequence of such a mutation will usually differ by one or more nucleotides or amino acids. The sequence changes may be substitutions, insertions, deletions, or a combination thereof. Techniques for mutagenesis of cloned genes are known in the art. Methods for site specific mutagenesis may be found in Gustin et al., Biotechniques 14:22, 1993; Barany, Gene 37:111-23, 1985; Colicelli et al., Mol. Gen. Genet. 199:537-9, 1985; and Sambrook et al., Molecular Cloning: A Laboratory Manual, CSH Press 1989, pp. 15.3-15.108 and all incorporated herein by reference. In summary, the invention relates to nucleic acid sequences capable of stimulating an innate immune response in a subject and variants or mutants thereof. Also, the invention encompasses the intermediatary RNAs encoded by the described nucleic acid sequences, as well as any resultant amino acid sequences encoded.

In some aspects, where the nucleotide sequence of the immunostimulatory plasmid varies from the sequences provided in SEQ ID NOs. 1, 2, 3, and 4 the CpG dinucleotides in the plasmid are preferably left intact. Alternatively, if the nucleotide sequence of the plasmid is altered such that a CpG dinucleotide is eliminated, the sequence of the plasmid may be altered at another location such that the total number of CpG dinucleotides in the plasmid remains the same. Further CpG dinucleotides in addition to those already present in the nucleotide sequences of pGCMB75.6 or pLacZMB75.6 may also be introduced into the plasmid. Thus, for example, the immunostimulatory plasmids described herein preferably comprise at least about 200, at least about 220, at least about 240, at least about 260, at least about 270, at least about 275, at least about 280, at least about 283, at least about 285, or at least about 288 CpG dinucleotides. For example, the immunostimulatory plasmid can comprise 283 CpG dinucleotides.

In some aspects, where the nucleotide sequence of the immunostimulatory plasmid varies from the sequences provided herein, the CpG motif types in the plasmid are varied to modulate the resultant activation of the cytosolic DNA surveillance molecules. For example, the number of immune stimulatory CpG motifs may be increased to increase the activation of specific cytosolic DNA surveillance molecules responsive to a specific threshold of immunostimulatory plasmid/DNA. By way of further example, the number of non-immune stimulatory CpG motifs may be increased to decrease the activation of specific cytosolic DNA surveillance molecules and/or increase activation of other DNA surveillance molecules.

In particular, the present invention relates to pharmaceutical formulations comprising any of the immunostimulatory plasmids or DNA sequences described herein and a pharmaceutically acceptable carrier.

B. Immunomodulator

Suitable immunomodulator compositions for use with the immunostimulatory plasmids described herein are described in U.S. Patent Application Publications Nos. 2012/0064151 A1 and 2013/0295167 A1 the contents of both of which are hereby incorporated by reference in their entirety.

The immunomodulator composition comprises a liposome delivery vehicle and at least one of the immunostimulatory plasmids, or DNA sequences, described herein.

A suitable liposome delivery vehicle comprises a lipid composition that is capable of delivering nucleic acid molecules to the tissues of the treated subject. A liposome delivery vehicle is preferably capable of remaining stable in a subject for a sufficient amount of time to deliver a nucleic acid molecule and/or a biological agent. For example, the liposome delivery vehicle is stable in the recipient subject for at least about five minutes, for at least about 1 hour, or for at least about 24 hours.

A liposome delivery vehicle of the present invention comprises a lipid composition that is capable of fusing with the plasma membrane of a cell to deliver a nucleic acid molecule into a cell. When the nucleic acid molecule encodes one or more proteins, the nucleic acid:liposome complex preferably has a transfection efficiency of at least about 1 picogram (pg) of protein expressed per milligram (mg) of total tissue protein per microgram (μg) of nucleic acid delivered. For example, the transfection efficiency of a nucleic acid: liposome complex can be at least about 10 pg of protein expressed per mg of total tissue protein per μg of nucleic acid delivered; or at least about 50 pg of protein expressed per mg of total tissue protein per μg of nucleic acid delivered. The transfection efficiency of the complex may be as low as 1 femtogram (fg) of protein expressed per mg of total tissue protein per μg of nucleic acid delivered, with the above amounts being more preferred.

A preferred liposome delivery vehicle of the present invention is between about 100 and 500 nanometers (nm) in diameter. For example, the liposome delivery vehicle can be between about 150 and 450 nm or between about 200 and 400 nm in diameter.

Suitable liposomes include any liposome, such as those commonly used in, for example, gene delivery methods known to those of skill in the art. Preferred liposome delivery vehicles comprise multilamellar vesicle (MLV) lipids and extruded lipids. Methods for preparation of MLVs are well known in the art. More preferred liposome delivery vehicles comprise liposomes having a polycationic lipid composition (i.e., cationic liposomes) and/or liposomes having a cholesterol backbone conjugated to polyethylene glycol. Exemplary cationic liposome compositions include, but are not limited to, N-[1-(2,3-dioleyloxy)propyl]-N,N,N-trimethylammonium chloride (DOTMA) and cholesterol, N-[1-(2,3-dioleoyloxy)propyl]-N,N,N-trimethylammonium chloride (DOTAP) and cholesterol, 1-[2-(oleoyloxy)ethyl]-2-oleyl-3-(2-hydroxyethyl)-imidazolinium chloride (DOTIM) and cholesterol, dimethyldioctadecylammonium bromide (DDAB) and cholesterol, and combinations thereof. A most preferred liposome composition for use as a delivery vehicle includes DOTIM and cholesterol.

A suitable nucleic acid molecule includes any of the immunostimulatory plasmids described herein. Coding nucleic acid sequences encode at least a portion of a protein or peptide, while non-coding sequence does not encode any portion of a protein or peptide. According to the present invention, “non-coding” nucleic acids can include regulatory regions of a transcription unit, such as a promoter region. The term, “empty vector” can be used interchangeably with the term “non-coding,” and particularly refers to a nucleic acid sequence in the absence of a protein coding portion, such as a plasmid vector without a gene insert. Expression of a protein encoded by the plasmids described herein is not required for inducing an immune response; therefore the plasmids need not contain any coding sequences operatively linked to a transcription control sequence. However, further advantages may be obtained (i.e., antigen-specific and enhanced immunity) by including in the composition nucleic acid sequence (DNA or RNA) which encodes an immunogen and/or a cytokine. Such a nucleic acid sequence encoding an immunogen and/or a cytokine may be included in the immunostimulatory plasmids described herein, or may be included in a separate nucleic acid (e.g., a separate plasmid) in the composition.

Complexing a liposome with the immunostimulatory plasmids, or immunostimulatory DNA sequence, described herein may be achieved using methods standard in the art or as described in U.S. Pat. No. 6,693,086, the contents of which are hereby incorporated by reference in their entirety. A suitable concentration of a plasmid to add to a liposome includes a concentration effective for delivering a sufficient amount of the plasmid into a subject such that a systemic immune response is elicited. For example, from about 0.1 μg to about 10 μg of plasmid can be combined with about 8 nmol liposomes, from about 0.5 μg to about 5 μg of plasmid can be combined with about 8 nmol liposomes, or about 1.0 μg of plasmid can be combined with about 8 nmol liposomes. The ratio of plasmid to lipid (μg plasmid:nmol lipid) in a composition can be at least about 1:1 plasmid:lipid by weight (e.g., 1 μg plasmid:1 nmol lipid). For example, the ratio of plasmid to lipids can be at least about 1:5, at least about 1:10, or at least about 1:20. Ratios expressed herein are based on the amount of cationic lipid in the composition, and not on the total amount of lipid in the composition. The ratio of plasmid to lipids in a composition of the invention is suitably from about 1:1 to about 1:80 plasmid:lipid by weight; from about 1:2 to about 1:40 plasmid:lipid by weight; from about 1:3 to about 1:30 plasmid:lipid by weight; or from about 1:6 to about 1:15 plasmid:lipid by weight.

C. Biological Agent

Any of the immunomodulator compositions described herein can further comprise at least one biological agent, in addition to the liposome delivery vehicle and at least one of the plasmids described herein.

Suitable biological agents are agents that are effective in preventing or treating diseases. Such biological agents include immune enhancer proteins, immunogens, vaccines, antimicrobials or any combination thereof. Suitable immune enhancer proteins are those proteins known to enhance immunity. By way of a non-limiting example, a cytokine, which includes a family of proteins, is a known immunity enhancing protein family. Suitable immunogens are proteins which elicit a humoral and/or cellular immune response such that administration of the immunogen to a subject mounts an immunogen-specific immune response against the same or similar proteins that are encountered within the tissues of the subject. An immunogen may include a pathogenic antigen expressed by a bacterium, a virus, a parasite or a fungus. Preferred antigens include antigens derived from organisms which cause an infectious disease in a subject. According to the present invention, an immunogen may be any portion of a protein, naturally occurring or synthetically derived, which elicits a humoral and/or cellular immune response. As such, the size of an antigen or immunogen may be as small as about 5-12 amino acids and as large as a full length protein, including any sizes in between. The antigen may be a multimer protein or fusion protein. The antigen may be a purified antigen. Alternatively, the immune enhancer protein or immunogen can be encoded by the immunostimulatory plasmid or by another nucleic acid included in the immunomodulator composition. Where the immune enhancer protein or immunogen is encoded by a nucleic acid molecule in the immunomodulator composition, the nucleic acid sequence encoding the immune enhancer protein or immunogen is operatively linked to a transcription control sequence, such that the immunogen is expressed in a tissue of a subject, thereby eliciting an immunogen-specific immune response in the subject, in addition to the non-specific immune response. Techniques to screen for immunogenicity, such as pathogen antigen immunogenicity or cytokine activity are known to those of skill in the art and include a variety of in vitro and in vivo assays.

Where the biological agent is a vaccine, the vaccine may include a live, infectious, viral, bacterial, or parasite vaccine or a killed, inactivated, viral, bacterial, or parasite vaccine, or subunit vaccine, or any vaccine known in the art. One or more vaccines may be used in combination with the immunomodulator composition of the present invention. Suitable vaccines include those known in the art for aquatic species.

The biological agent can be an antimicrobial. Suitable antimicrobials include: quinolones, preferably fluoroquinolones, β-lactams, and macrolide-lincosamide-streptogramin (MLS) antibiotics.

Suitable quinolones include benofloxacin, binfloxacin, cinoxacin, ciprofloxacin, clinafloxacin, danofloxacin, difloxacin, enoxacin, enrofloxacin, fleroxacin, gemifloxacin, ibafloxacin, levofloxacin, lomefloxacin, marbofloxacin, moxifloxacin, norfloxacin, ofloxacin, orbifloxacin, pazufloxacin, pradofloxacin, perfloxacin, sarafloxacin, sparfloxacin, temafloxacin, and tosufloxacin. Preferred fluoroquinolones include ciprofloxacin, danofloxacin, enrofloxacin, moxifloxacin, and pradofloxacin. Suitable naphthyridones include nalidixic acid.

Suitable β-lactams include penicillins (e.g., amoxicillin, ampicillin, azlocillin, benzathine penicillin, benzylpenicillin, carbenicillin, cloxacillin, co-amoxiclav [i.e. amoxicillin/clavulanic acid], dicloxacillin, flucloxacillin, methicillin, mezlocillin, nafcillin, oxacillin, phenoxymethylpenicillin, piperacillin, procaine penicillin, temocillin, and ticarcillin); cephalosporins (e.g., cefaclor, cefalonium, cefamandole, cefapririn, cefazolin, cefepime, cefixime, cefotaxime, cefoxitin, cefpirome, cefpodoxime, cefquinome, ceftazidime, ceftiofur, ceftriaxone, cefuroxime, cephalexin, cephalothin, and defotetan); carbapenems and penems (e.g., doripenem, ertapenem, faropenem, imipenem, and meropenem); monobactams (e.g., aztreonam, nocardicin A, tabtoxinine-β-lactam, and tigemonam); and β-lactamase inhibitors (e.g., clavulanic acid, sulbactam, and tazobactam). Preferred β-lactams include cephalosporins, in particular, cefazolin.

Suitable MLS antibiotics include clindamycin, lincomycin, pirlimycin, and any macrolide antibiotic. A preferred lincosamide antibiotic is pirlimycin.

Other antimicrobials include aminoglycosides, clopidol, dimetridazoles, erythromycin, framycetin, furazolidone, halofuginone, 2-pyridones, robenidine, sulfonamides, tetracyclines, trimethoprim, various pleuromutilins (e.g., tiamulin and valnemulin), and various streptomycin (e.g., monensin, narasin, and salinomycin).

II. Methods

An object of the present invention is to provide immunomodulator compositions, immunostimulatory plasmids (or DNA sequence), and methods that stimulate immunity and provide protective immunity to uninfected subjects, protective immunity to infected subjects, enhanced immunity to uninfected subjects, enhanced immunity to infected subjects, therapeutic immunity to infected subjects, or combinations thereof. As such, the compositions of the invention may be used to prophylactically immunize a subject or be used to treat a subject. The methods described herein include administrating an immunostimulatory plasmid, or DNA sequence, described herein to a subject, and stimulating the immune system in the subject.

A. Methods of Stimulating the Immune System of a Subject

The present invention is related to methods of stimulating, or enhancing, the immune system, in a recipient subject. The methods comprise administering to a subject an effective amount of an immunomodulator composition described herein. In some aspects, the immunomodulator composition stimulates an immune response in a recipient subject and the immune response helps fight off infection. In some aspects, the immunomodulator composition activates cytosolic DNA surveillance molecules. In some aspects, the immunomodulator composition enhances the operation of at least one biological agent such as a vaccine, when administered prior to such a vaccine, co-administered with a vaccine, administered post vaccination, or mixed with the vaccine. In some aspects, the methods provide new treatment strategies for protecting recipient subjects from infectious diseases and treating populations having infectious disease. In some aspects, the methods provide a more rapid, a longer and better protection against a disease when the immunomodulator is used in combination with a vaccine, compared to use of the vaccine without the immunomodulator composition.

An immune response can be activated in a recipient subject by administering an effective amount of an immunomodulator composition, which includes any of the liposome delivery vehicles described herein, any of the immunostimulatory plasmids (or DNA sequences) described herein, and optionally any of the biological agents described herein. It is contemplated that the biological agent may be mixed with or co-administered with the immunomodulator or independently thereof. Independent administration may be prior to or after administration of the immunomodulator. It is also contemplated that more than one administration of the immunomodulator or biological agent may be used. Furthermore, more than one biological agent may be co-administered with the immunomodulator, administered prior to the immunomodulator, administered after administration of the immunomodulator, or concurrently with the immunomodulator.

B. Methods of Improving Survivability of a Subject

The present invention is related to methods of improving survivability of a member of an aquatic species. Methods of improving survivability include administering to a subject an effective amount of an immunomodulator composition described herein. In some aspects, the methods provide improved survivability of recipient subjects compared to subject not receiving the immunomodulator composition.

C. Methods of Improving Production

The present invention is related to methods of improving production of a member of an aquatic species raised in aqua culture. Methods of improving production include administering to a subject an effective amount of an immunomodulator composition described herein. In some aspects, the methods provide improved production of recipient subjects compared to subjects not receiving the immunomodulator composition. Methods of assessing improved production are known in the art. A skilled artisan will recognize that improved production may be measured comparing the health, weight, size, meat quality, and other parameters between subjects receiving the immunomodulator composition and those subjects not receiving the immunomodulator composition.

D. Methods of Improving Feed Utilization

The present invention is related to methods of improving feed utilization of a member of an aquatic species raised in aqua culture. Methods of improving feed utilization include administering to a subject an effective amount of an immunomodulator composition described herein. In some aspects, the methods provide improved feed utilization of recipient subjects compared to subjects not receiving the immunomodulator composition. Methods of assessing feed utilization are known in the art. A skilled artisan will recognize that improved feed utilization may be measured comparing the health, weight, size, meat quality, and other parameters between subjects receiving the immunomodulator composition and those subjects not receiving the immunomodulator composition.

E. Methods of Improving Survival Rate

The present invention is related to methods of improving survival rate of a member of an aquatic species raised in aqua culture. Methods of improving survival rate include administering to a subject an effective amount of an immunomodulator composition described herein. In some aspects, the methods provide improved survival rate of recipient subjects compared to subjects not receiving the immunomodulator composition. Methods of assessing improved survival rate are known in the art. A skilled artisan will recognize that improved survival rate may be measured comparing the health, weight, size, meat quality, and other parameters between subjects receiving the immunomodulator composition and those subjects not receiving the immunomodulator composition.

An effective amount of any of the immunomodulator compositions described herein may be administered to a subject. The effective amount is sufficient to activate an immune response in the recipient subject. Methods of measuring such activation are known in the art. Also, a skilled artisan will recognize that the effective amount will depend upon age, weight, species of the subject and stage of infection, as well as other factors known in the art. Suitable effective amounts may range from about 0.1 μg to 1,000 μg per subject. In some aspects, the effective amount may range from about 0.1 μg to about 10 μg, from about 0.1 μg to about 5 μg, from about 0.5 μg to about 5 μg, from about 0.25 μg to about 5 μg, from about 0.05 μg to about 10 μg, from about 5 μg to about 15 μg, from about 10 μg to about 15 μg, from about 10 μg to about 20 μg, from about 20 μg to about 30 μg, from about 30 μg to about 40 μg, from about 40 μg to about 50 μg, from about 50 μg to about 70 μg, from about 70 μg to about 90 μg, from about 50 μg to about 100 μg, from about 100 μg to about 150 μg, from about 150 μg to about 200 μg, from about 200 μg to about 250 μg, from about 250 μg to about 300 μg, from about 300 μg to about 350 μg, from about 350 μg to about 400 μg, from about 400 μg to about 450 μg, from about 450 μg, to about 500 μg, from about 500 μg to about 550 μg, from about 550 μg to about 600 μg, from about 600 μg to about 650 μg, from about 650 μg to about 700 μg, from about 700 μg to about 750 μg, from about 750 μg to about 800 μg, from about 800 μg to about 850 μg, from about 850 μg to about 900 μg, from about 900 μg to about 950 μg, from about 950 μg to about 1000 μg. Preferably, in some aspects, the effective amount ranges from about 0.5 μg to about 10 μg.

F. Conditions for Use

The methods of the invention activate an immune response in a subject such that the subject is protected from a disease that is amenable to elicitation of an immune response. As used herein, the phrase “protected from a disease” refers to reducing the symptoms of the disease; reducing the occurrence of the disease; reducing the clinical or pathologic severity of the disease; or reducing shedding of a pathogen causing a disease. Protecting a subject can refer to the ability of a therapeutic composition of the present invention, when administered to a subject, to prevent a disease from occurring, cure, and/or alleviate or reduce disease symptoms, clinical signs, pathology, or causes. As such, protecting a subject from a disease encompasses both preventing disease occurrence (prophylactic treatment) and treating a subject that has a disease (therapeutic treatment). The term “disease” refers to any deviation from the normal health of a subject and includes a state when disease symptoms are present, as well as conditions in which a deviation (e.g., infection, gene mutation, genetic defect, etc.) has occurred, but symptoms are not yet manifested.

Methods of the invention may be used for the prevention of disease, stimulation of effector cell immunity against disease, elimination of disease, alleviation of disease, and prevention of a secondary disease resulting from the occurrence of a primary disease.

In some aspects, methods described herein may be used to improve the innate immune response of the subject when co-administered with a vaccine versus administration of the vaccine by itself. In some aspects, methods described herein may be used to improve the acquired immune response of the subject when co-administered with a vaccine versus administration of the vaccine by itself. Generally a vaccine once administered does not immediately protect the subject as it takes time to stimulate acquired immunity. The term “improve” refers, in the present invention, to elicitation of an innate immune response in the subject until the vaccine starts to protect the subject and/or to prolong the period of protection, via acquired immunity, given by the vaccine.

In some aspects, methods of the invention include administering the composition to protect against infection of a wide variety of pathogens. The composition administered may or may not include a specific antigen to elicit a specific response. It is contemplated that the methods of the invention will protect the recipient subject from disease resulting from infectious microbial agents including, without limitation, viruses, bacteria, fungi, and parasites. A skilled artisan will recognize and appreciate that a immunomodulator composition, as described herein, is effective against numerous infectious agents, which are too numerous to list. The infectious agents provided herein are provided for exemplary purposes and are provided without limitation of the scope of use.

G. Administration

A variety of administration routes are available. The particular mode selected will depend, of course, upon the particular biological agents selected, the age and general health status of the subject, the particular condition being treated and the dosage required for therapeutic efficacy. The methods of this invention may be practiced using any mode of administration that produces effective levels of activation of an immune response without causing clinically unacceptable adverse effects. The compositions may conveniently be presented in unit dosage form and may be prepared by any of the methods well known in the art.

The immunomodulator composition may be administered intravenously, intramuscularly, intradermally, intraperitoneally, subcutaneously, by spray, in ovo, orally, intraocularly, intratracheally, intranasally, by immersion, uptake via skin or gills during bath, or by other methods known in the art. In one aspect, the immunomodulator is administered subcutaneously. In another aspect, the immunomodulator may be administered intramuscularly. In another aspect, the immunomodulator is administered as a spray. In another aspect, the immunomodulator may be administered orally. In another aspect, the immunomodulator may be administered subcutaneously.

In one respect, the immunomodulator may be administered by itself to the subject prior to challenge (or infection). In another aspect, the immunomodulator may be administered by itself to the subject post challenge (or infection). In another aspect, the immunomodulator may be administered by itself to the subject at the same time as challenge (or infection).

In some aspects, the immunomodulator composition may be co-administered at the same time as the vaccination prior to challenge. In some aspects, the immunomodulator composition may be co-administered at the same time as the vaccination at the same time as challenge (or infection). In some aspects, the co-administration may include administering the vaccine and immunomodulator in the same general location on the subject at two different sites next to each other (i.e., injections next to each other at the neck of the subject), on opposing sides of the subject at the same general location (i.e., one on each side of the neck), or on different locations of the same subject. In some aspects, the immunomodulator composition can be administered prior to vaccination and challenge. In some aspects, the immunomodulator composition may be administered after vaccination but prior to challenge. The immunomodulator composition can be administered after challenge to a subject that has been vaccinated prior to challenge (or infection).

A skilled artisan will recognize that administration routes may vary depending upon the subject and the health or state of the subject. The administration routes provided for are for exemplary purposes and are provided without limitation.

Vaccination may be performed at any age. The vaccine may be administered subcutaneously, by spray, orally, intraocularly, intratracheally, intranasally, in ovo, by immersion or by other methods know in the art. Oral vaccines may be administered in feed or water. Further, it is contemplated that the methods of the invention may be used based on routine vaccination schedules.

The immunomodulator composition may also be administered subcutaneously, by intraocularly, intratracheally, intranasally, in ovo, or by other methods known in the art. For example, the immunomodulator composition can be administered in ovo. Alternatively, the immunomodulator composition can be administered as a spray.

The administration to the egg may be prior to challenge (or infection) or post challenge.

The immunomodulator can be administered to a subject from about 1 to about 14 days prior to challenge or from about 1 to about 14 days post challenge. For example, the immunomodulator can be administered from about 1 to about 7 days prior to challenge or from about 1 to about 7 days post challenge. The immunomodulator is suitably administered 1, 2, 3, 4, 5, 6, 7 days prior to challenge or 1, 2, 3, 4, 5, 6, 7 days post challenge.

Other delivery systems may include time-release, delayed release, or sustained release delivery systems. Such systems can avoid repeated administrations of the compositions therefore increasing convenience. Many types of release delivery systems are available and known to those of ordinary skill in the art. They include polymer based systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109.

Delivery systems also include non-polymer systems that are lipids including sterols such as cholesterol, cholesterol esters, and fatty acids or neutral fats such as mono-, di-, and tri-glycerides; hydrogel release systems; silastic systems; peptide based systems; wax coatings; compressed tablets using conventional binders and excipients; partially fused implants; and the like. Specific examples include, but are not limited to, erosional systems in which an agent of the invention is contained in a form within a matrix such as those described in U.S. Pat. Nos. 4,452,775, 4,675,189, and 5,736,152, and diffusional systems in which an active component permeates at a controlled rate from a polymer such as described in U.S. Pat. Nos. 3,854,480, 5,133,974, and 5,407,686. In addition, pump-based hardware delivery systems can be used, some of which are adapted for implantation.

As various changes could be made in the above composition, products, and methods without departing from the scope of the invention, it is intended that all matter contained in the above description and in the examples given below shall be interpreted as illustrative and not in a limiting sense.

### Definitions

The term “effective amount” refers to the amount necessary or sufficient to realize a desired biologic effect. For example, an effective amount of immunomodulator for treating or preventing an infectious disease is that amount necessary to cause the development of an immune response upon exposure to the microbe, thus causing a reduction in the amount of microbe within the subject and preferably the eradication of the microbe. The effective amount for any particular application can vary depending on such factors as the disease or condition being treated, the size of the subject, or the severity of the disease or condition. One of ordinary skill in the art can empirically determine the effective amount of immunomodulator without necessitating undue experimentation.

The term “cytokine” refers to an immune enhancing protein family. The cytokine family includes hematopoietic growth factor, interleukins, interferons, immunoglobulin superfamily molecules, tumor necrosis factor family molecules and chemokines (i.e. proteins that regulate the migration and activation of cells, particularly phagocytic cells). Exemplary cytokines include, without limitation, interleukin-2 (IL-2), interleukin-12 (IL-12), interleukin-15 (IL-15), interleukin-18 (IL-18), interferon-α (IFNα), and interferon-γ (IFNγ).

The term “elicit” can be used interchangeably with the terms activate, stimulate, generate or upregulate.

The term “eliciting an immune response” in a subject refers to specifically controlling or influencing the activity of the immune response, and can include activating an immune response, upregulating an immune response, enhancing an immune response and/or altering an immune response (such as by eliciting a type of immune response which in turn changes the prevalent type of immune response in a subject from one which is harmful or ineffective to one which is beneficial or protective).

The term “operatively linked” refers to linking a nucleic acid molecule to a transcription control sequence in a manner such that the molecule is able to be expressed when transfected (i.e., transformed, transduced or transfected) into a host cell. Transcriptional control sequences are sequences which control the initiation, elongation, and termination of transcription. Particularly important transcription control sequences are those which control transcription initiation, such as promoter, enhancer, operator and repressor sequences. A variety of such transcription control sequences are known to those skilled in the art. Preferred transcription control sequences include those which function in avian, fish, mammalian, bacteria, viral, plant, and insect cells. While any transcriptional control sequences may be used with the invention, the sequences may include naturally occurring transcription control sequences naturally associated with a sequence encoding an immunogen or immune stimulating protein.

The terms “nucleic acid molecule” and “nucleic acid sequence” can be used interchangeably and include DNA, RNA, or derivatives of either DNA or RNA. The terms also include oligonucleotides and larger sequences such as plasmids, such as the immunostimulatory plasmids described herein, and including both nucleic acid molecules that encode a protein or a fragment thereof, and nucleic acid molecules that comprise regulatory regions, introns, or other non-coding DNA or RNA. Typically, an oligonucleotide has a nucleic acid sequence from about 1 to about 500 nucleotides, and more typically, is at least about 5 nucleotides in length. The nucleic acid molecule can be derived from any source, including mammalian, fish, bacterial, insect, viral, plant, synthetic sources or combinations thereof. A nucleic acid molecule can be produced by methods commonly known in the art such as recombinant DNA technology (e.g., polymerase chain reaction (PCR), amplification, cloning) or chemical synthesis. Nucleic acid molecules include natural nucleic acid molecules and homologues thereof, including, but not limited to, natural allelic variants and modified nucleic acid molecules in which nucleotides have been inserted, deleted, substituted, or inverted in such a manner that such modifications do not substantially interfere with the nucleic acid molecule's ability to elicit an immune response useful in the methods of the present invention. A nucleic acid homologue may be produced using a number of methods known to those skilled in the art (see, for example, Sambrook et al., Molecular Cloning: A Laboratory Manual, Cold Spring Harbor Labs Press, 1989), which is incorporated herein by reference.

The terms “selectable marker” and “selectable marker gene” refer to a gene that encodes a product that protects the organism in which the gene is expressed from a selective agent (e.g., an antibiotic) or a condition that would normally kill the organism or inhibit its growth. Selectable marker genes are most commonly antibiotic resistance genes (e.g., kanamycin resistance genes, ampicillin resistance genes, chloramphenicol resistance genes, tetracycline resistance genes, etc.). Thus, for example, when E. coli cells are subjected to a transformation procedure to introduce a plasmid encoding a kanamycin resistance gene and then grown on or in media containing kanamycin, only the E. coli cells that have successfully taken up the plasmid and expressed the kanamycin resistance gene will survive. The terms “selectable marker” and “selectable marker gene” also include genes that code for enzymes involved in the synthesis of a compound that is essential for the growth of an organism. When introduced into an auxotrophic organism that is unable to synthesize the essential compound, such genes allow the organism to grow in a medium that has been supplemented with the essential compound. For example, bacterial cells that are auxotrophic for the amino acid lysine due to a mutation in or the absence of an enzyme involved in lysine biosynthesis normally are unable to grown on media that has not been supplemented with lysine. When such bacteria are subjected to a transformation procedure to introduce a plasmid encoding the enzyme involved in lysine biosynthesis, the bacteria that have successfully taken up the plasmid and expressed the enzyme will survive when grown on media that has not been supplemented with lysine. The terms “selectable marker” and “selectable marker gene” further include genes that allow for poison/antidote selection. For example, the ccdB gene encodes a protein that binds to DNA gyrase, an essential enzyme for cell division. Upon binding to DNA gyrase, the ccdB gene product impairs gene replication and induces cell death. Thus, bacterial expressing the ccdB gene product cannot survive. The ccdA gene encodes a protein (the “antidote”) that acts as a natural inhibitor of the ccdB gene product. Thus, when bacteria having the ccdB gene in their bacterial genome are subjected to a transformation procedure to introduce a plasmid encoding the ccdA gene product, only the cells that successfully take up the plasmid and express the ccdA gene will survive.

The terms “screenable marker” and “screenable marker gene” refer to a gene that encodes a product that allows an observer to distinguish between cells expressing the screenable marker gene and cells that are not expressing the screenable marker gene. Screenable marker gene systems are well known in the art and include, for example, lacZ genes and genes encoding fluorescent proteins such as green fluorescent protein (GFP), yellow fluorescent protein (YFP), red fluorescent protein (RFP), blue fluorescent protein (BFP), or cyan fluorescent protein (CFP).

As used herein, the term “improving production” refers to an improvement in a measurement such as the health, weight, size, meat quality, or other parameter between subjects receiving the immunomodulator composition and those subjects not receiving the immunomodulator composition.

As used herein, the term “improving survivability” refers to improved survival of recipient subjects compared to subject not receiving the immunomodulator composition.

As used herein, the term “subject” refers to a member of the aquatic species, including any of the considerable varieties of aquatic species. Subjects may be any member of aquatic species, whether domestic or wild, and may be commercially reared for breeding, meat or egg production. Exemplary aquatic species include, without limitation, any species of fish, crustaceans, molluscs, living in freshwater or saltwater. Representative species include, without limitation, fish including any member of the Phylum Chordata, Sub Phylum Vertebrata, and Super Class Pisces. By further example, without limitation, such fish include catfish, channel catfish, black bullhead, yellow bullhead, brown bullhead, carp, crucian carp, trout, rainbow trout, brown trout, speckled brook trout, salmon, atlantic salmon, coho salmon, chinooK or king salmon, tench, roach, pike, pike-perch, dover sole, turbot, yellowtail, bass, smallmouth bass, largemouth bass, striped bass, stripers, milkfish, tilapia, gray mullet, eels, cod, and any other variety or aquatic species with which the present invention may be practiced will be apparent to those skilled in the art. In some aspects, subjects may be diagnosed with an infectious disease, may be at risk for an infectious disease, or may be experiencing an infectious disease. Subjects may be of any age including in ovo, new born, adolescence, adult, middle age, or elderly.

TABLE 1
Plasmid DNA Sequences

| | SEQ | |
|---|---|---|
| | ID | |
| Plasmid | NO. | SEQUENCE |
| pMB75.6 | 2 | ctaaattgtaagcgttaatattttgttaaaattcgcgttaaatttttgttaaatcagctcattttttaaccaata |
| | | ggccgaaatcggcaaaatcccttataaatcaaaagaatagaccgagatagggttgagtgttgttcca |
| | | gtttggaacaagagtccactattaaagaacgtggactccaacgtcaaagggcgaaaaaccgtctatc |
| | | agggcgatggcccactacgtgaaccatcaccctaatcaagttttttggggtcgaggtgccgtaaagc |
| | | actaaatcggaaccctaaagggagcccccgatttagagcttgacggggaaagccggcgaacgtg |
| | | gcgagaaaggaagggaagaaagcgaaaggagcgggcgctagggcgctggcaagtgtagcggt |
| | | cacgctgcgcgtaaccaccacacccgccgcgcttaatgcgccgctacagggcgcgtcccattcgc |
| | | cattcaggctgcgcaactgttgggaagggcgatcggtgcgggcctcttcgctattacgccagctgg |
| | | cgaaagggggatgtgctgcaaggcgattaagttgggtaacgccagggttttcccagtcacgacgtt |
| | | gtaaaacgacggccagtgagcgcgcgtaatacgactcactatagggcgaattgggtaccgggccc |
| | | cccctcgagcaggatctatacattgaatcaatattggcaattagccatattagtcattggttatatagcat |
| | | aaatcaatattggctattggccattgcatacgttgtatctatatcataatatgtacatttatattggctcatg |
| | | tccaatatgaccgccatgttgacattgattattgactagttattaatagtaatcaattacggggtcattagt |
| | | tcatagcccatatatggagttccgcgttacataacttacggtaaatggcccgcctggctgaccgccca |
| | | acgacccccgcccattgacgtcaataatgacgtatgttcccatagtaacgccaatagggactttccat |
| | | tgacgtcaatgggtggagtatttacggtaaactgcccacttggcagtacatcaagtgtatcatatgcca |
| | | agtccgccccctattgacgtcaatgacggtaaatggcccgcctggcattatgcccagtacatgacctt |
| | | acgggactttcctacttggcagtacatctacgtattagtcatcgctattaccatggtgatgcggttttgg |
| | | cagtacatcaatgggcgtggatagcggtttgactcacggggatttccaagtctccaccccattgacgt |
| | | caatgggagtttgttttggcaccaaaatcaacgggactttccaaaatgtcgtaacaactccgccccatt |
| | | gacgcaaatgggcggtaggcgtgtacggtgggaggtctatataagcagagctcgtttagtgaaccg |
| | | tcagatcgcctggagacgccatccacgctgttttgacctccatagaagacaccgggaccgatccag |
| | | cctcccctcgaagccgatctgataacggtaccgataagctggcggccgattaagctacagaagttg |
| | | gtcgtgaggcactgggcaggtaagtatcaaggttacaagacaggtttaaggagaccaatagaaact |
| | | gggcttgtcgagacagagaagactcttgcgtttctgataggcacctattggtcttactgacatccacttt |
| | | gcctttctctccacaggtgtccactcccaggttcaattacagctcttaagcagccgcaagcttgatatc |
| | | gaattcctgcagcccgggggatccactagttctagagcggccgccaccgcggtggagctcgaatt |
| | | atcagatcgattaataactatgctcaaaaattgtgtacctttagctttttaatttgtaaaggggttaataag |
| | | gaatatttgatgtatagtgccttgactagagatcataatcagccataccacatttgtagaggttttacttg |
| | | ctttaaaaaacctcccacacctccccctgaacctgaaacataaaatgaatgcaattgttgttgttaactt |
| | | gtttattgcagcttataatggttacaaataaagcaatagcatcacaaatttcacaaataaagcatttttttc |
| | | actgcattctagttgtggtttgtccaaactcatcaatgtatcttatcatgtctggatcatcagatctgccgg |
| | | tctccctatagtgagtcgtattaatttcgataagccaggttaacctgcattaatgaatcggccaacgcg |
| | | cggggagaggcggtttgcgtattgggcgctcttccgcttcctcgctcactgactcgctgcgctcggt |
| | | cgttcggctgcggcgagcggtatcagctcactcaaaggcggtaatacggttatccacagaatcagg |
| | | ggataacgcaggaaagaacatgtgagcaaaaggccagcaaaaggccaggaaccgtaaaaaggc |
| | | cgcgttgctggcgtttttccataggctccgcccccctgacgagcatcacaaaaatcgacgctcaagt |
| | | cagaggtggcgaaacccgacaggactataaagataccaggcgtttccccctggaagctccctcgt |
| | | gcgctctcctgttccgaccctgccgcttaccggatacctgtccgcctttctcccttcgggaagcgtgg |
| | | cgctttctcatagctcacgctgtaggtatctcagttcggtgtaggtcgttcgctccaagctgggctgtgt |
| | | gcacgaaccccccgttcagcccgaccgctgcgccttatccggtaactatcgtcttgagtccaacccg |
| | | gtaagacacgacttatcgccactggcagcagccactggtaacaggattagcagagcgaggtatgta |
| | | ggcggtgctacagagttcttgaagtggtggcctaactacggctacactagaagaacagtatttggtat |
| | | ctgcgctctgctgaagccagttaccttcggaaaaagagttggtagctcttgatccggcaaacaaacc |
| | | accgctggtagcggtggtttttttgtttgcaagcagcagattacgcgcagaaaaaaaggatctcaaga |
| | | agatcctttgatcttttctacggggtctgacgctcagtggaacgaaaactcacgttaagggattttggtc |
| | | atgagcgcgcctaggcttttgcaaagatcgatcaagagacaggatgaggatcgtttcgcatgattga |
| | | acaagatggattgcacgcaggttctccggccgcttgggtggagaggctattcggctatgactgggc |
| | | acaacagacaatcggctgctctgatgccgccgtgttccggctgtcagcgcaggggcgcccggttct |
| | | ttttgtcaagaccgacctgtccggtgccctgaatgaactgcaagacgaggcagcgcggctatcgtg |
| | | gctggccacgacgggcgttccttgcgcagctgtgctcgacgttgtcactgaagcgggaagggact |
| | | ggctgctattgggcgaagtgccggggcaggatctcctgtcatctcaccttgctcctgccgagaaagt |
| | | atccatcatggctgatgcaatgcggcggctgcatacgcttgatccggctacctgcccattcgaccac |
| | | caagcgaaacatcgcatcgagcgagcacgtactcggatggaagccggtcttgtcgatcaggatgat |
| | | ctggacgaagagcatcaggggctcgcgccagccgaactgttcgccaggctcaaggcgagcatgc |
| | | ccgacggcgaggatctcgtcgtgacccatggcgatgcctgcttgccgaatatcatggtggaaaatg |
| | | gccgcttttctggattcatcgactgtggccggctgggtgtggcggaccgctatcaggacatagcgtt |
| | | ggctacccgtgatattgctgaagagcttggcggcgaatgggctgaccgcttcctcgtgctttacggta |
| | | tcgccgctcccgattcgcagcgcatcgccttctatcgccttcttgacgagttcttctgagcgggactct |
| | | ggggttcgaaatgaccgaccaagcgacgcccaacctgccatcacgagatttcgattccaccgccg |
| | | ccttctatgaaaggttgggcttcggaatcgttttccgggacgccggctggatgatcctccagcgcgg |
| | | ggatctcatgctggagttcttcgcccaccctaggcgcgctcatgagcggatacatatttgaatgtattt |
| | | agaaaaataaacaaataggggttccgcgcacatttccccgaaaagtgccac |
| pGCMB75.6 | 1 | tgaccgcccaacgacccccgcccattgacgtcaataatgacgtatgttcccatagtaacgccaatag |
| | | ggactttccattgacgtcaatgggtggagtatttacggtaaactgcccacttggcagtacatcaagtgt |
| | | atcatatgccaagtccgccccctattgacgtcaatgacggtaaatggcccgcctggcattatgccca |
| | | gtacatgaccttacgggactttcctacttggcagtacatctacgtattagtcatcgctattaccatggtg |
| | | atgcggttttggcagtacatcaatgggcgtggatagcggtttgactcacggggatttccaagtctcca |
| | | ccccattgacgtcaatgggagtttgttttggcaccaaaatcaacgggactttccaaaatgtcgtaacaa |
| | | ctccgccccattgacgcaaatgggcggtaggcgtgtacggtgggaggtctatataagcagagctcg |
| | | tttagtgaaccgtcagatcgcctggagacgccatccacgctgttttgacctccatagaagacaccgg |
| | | gaccgatccagcctcccctcgaagccgatctgataacggtaccgataagctggcggccgattaagc |
| | | tacagaagttggtcgtgaggcactgggcaggtaagtatcaaggttacaagacaggtttaaggagac |
| | | caatagaaactgggcttgtcgagacagagaagactcttgcgtttctgataggcacctattggtcttact |
| | | gacatccactttgcctttctctccacaggtgtccactcccaggttcaattacagctcttaagcagccgc |
| | | aagcttgatatcgaattcctgcagcccgggggatccactagttctagagcggccgccaccgcggtg |
| | | gagctcgaattatcagatcgattaataactatgctcaaaaattgtgtacctttagctttttaatttgtaaag |
| | | gggttaataaggaatatttgatgtatagtgccttgactagagatcataatcagccataccacatttgtag |
| | | aggttttacttgctttaaaaaacctcccacacctccccctgaacctgaaacataaaatgaatgcaattgt |
| | | tgttgttaacttgtttattgcagcttataatggttacaaataaagcaatagcatcacaaatttcacaaataa |
| | | agcatttttttcactgcattctagttgtggtttgtccaaactcatcaatgtatcttatcatgtctggatcatca |
| | | gatctgccggtctccctatagtgagtcgtattaatttcgataagccaggttaacctgcattaatgaatcg |
| | | gccaacgcgcggggagaggcggtttgcgtattgggcgctcttccgcttcctcgctcactgactcgct |
| | | gcgctcggtcgttcggctgcggcgagcggtatcagctcactcaaaggcggtaatacggttatccac |
| | | agaatcaggggataacgcaggaaagaacatgtgagcaaaaggccagcaaaaggccaggaaccg |
| | | taaaaaggccgcgttgctggcgtttttccataggctccgcccccctgacgagcatcacaaaaatcga |
| | | cgctcaagtcagaggtggcgaaacccgacaggactataaagataccaggcgtttccccctggaag |
| | | ctccctcgtgcgctctcctgttccgaccctgccgcttaccggatacctgtccgcctttctcccttcggg |
| | | aagcgtggcgctttctcatagctcacgctgtaggtatctcagttcggtgtaggtcgttcgctccaagct |
| | | gggctgtgtgcacgaaccccccgttcagcccgaccgctgcgccttatccggtaactatcgtcttgag |
| | | tccaacccggtaagacacgacttatcgccactggcagcagccactggtaacaggattagcagagc |
| | | gaggtatgtaggcggtgctacagagttcttgaagtggtggcctaactacggctacactagaagaaca |
| | | gtatttggtatctgcgctctgctgaagccagttaccttcggaaaaagagttggtagctcttgatccggc |
| | | aaacaaaccaccgctggtagcggtggtttttttgtttgcaagcagcagattacgcgcagaaaaaaag |
| | | gatctcaagaagatcctttgatcttttctacggggtctgacgctcagtggaacgaaaactcacgttaag |
| | | ggattttggtcatgggcgcgcctaggcttttgcaaagatcgatcaagagacaggatgaggatcgtttc |
| | | gcagcttttcattctgactgcaacgggcaataagtctctgtgtggattaaaaaaagagtgtctgatagc |
| | | agcttctgaactggttacctgccgtgagtaaattaaaattttattgacttaggtcactaaggcgccttgc |
| | | gctgaggttgcgtcgtgatatcatcagggcagaccggttacatccccctaacaagctgtataaagag |
| | | aaatactatctcattggcgttgcccgcacctgacagtgcgacgttgggctgcgtccgtcgaccaacg |
| | | gtaccgaggtaacagcccaatctatccatgatctcggccaggccgggtcggccgttatgcagcccg |
| | | gctcgggtatgaagccattaaggagccgacccagcgcgaccgggcggccggtcacgctgcctct |
| | | gctgaagcctgcctgtcactccctgcgcggcgtacccgccgttctcatcgagtaggctccggatcg |
| | | cgaccccggacgggccctgggcccaggagcggcctatgacaaatgccgggtagcgatccggca |
| | | ttcagcattgactgcgcacggatccagtccttgcaggagccttatgccgaccgtagcaaaaaatgag |
| | | cccgagccgatcgcgagttgtgatccggtcccgccgattgccggtcgcgatgacggtcctgtgtaa |
| | | gcgttatcgttaccaattgtttaagaagtatatacgctacgaggtacttgataacttctgcgtagcatac |
| | | atgaggttttgtataaaaatggcgggcgatatcaacgcagtgtcagaaatccgaaacagtctgcggg |
| | | actctggggttcgaaatgaccgaccaagcgacgcccaacctgccatcacgagatttcgattccacc |
| | | gccgccttctatgaaaggttgggcttcggaatcgttttccgggacgccggctggatgatcctccagc |
| | | gcggggatctcatgctggagttcttcgcccaccctaggcgcgctcatgagcggatacatatttgaat |
| | | gtatttagaaaaataaacaaataggggttccgcgcacatttccccgaaaagtgccacctaaattgtaa |
| | | gcgttaatattttgttaaaattcgcgttaaatttttgttaaatcagctcattttttaaccaataggccgaaatc |
| | | ggcaaaatcccttataaatcaaaagaatagaccgagatagggttgagtgttgttccagtttggaacaa |
| | | gagtccactattaaagaacgtggactccaacgtcaaagggcgaaaaaccgtctatcagggcgatg |
| | | gcccactacgtgaaccatcaccctaatcaagttttttggggtcgaggtgccgtaaagcactaaatcgg |
| | | aaccctaaagggagcccccgatttagagcttgacggggaaagccggcgaacgtggcgagaaag |
| | | gaagggaagaaagcgaaaggagcgggcgctagggcgctggcaagtgtagcggtcacgctgcg |
| | | cgtaaccaccacacccgccgcgcttaatgcgccgctacagggcgcgtcccattcgccattcaggct |
| | | gcgcaactgttgggaagggcgatcggtgcgggcctcttcgctattacgccagctggcgaaagggg |
| | | gatgtgctgcaaggcgattaagttgggtaacgccagggttttcccagtcacgacgttgtaaaacgac |
| | | ggccagtgagcgcgcgtaatacgactcactatagggcgaattgggtaccgggccccccctcgagc |
| | | aggatctatacattgaatcaatattggcaattagccatattagtcattggttatatagcataaatcaatatt |
| | | ggctattggccattgcatacgttgtatctatatcataatatgtacatttatattggctcatgtccaatatgac |
| | | cgccatgttgacattgattattgactagttattaatagtaatcaattacggggtcattagttcatagcccat |
| | | atatggagttccgcgttacataacttacggtaaatggcccgcctggc |
| pLacZMB75.6 | 4 | tgaccgcccaacgacccccgcccattgacgtcaataatgacgtatgttcccatagtaacgccaatag |
| | | ggactttccattgacgtcaatgggtggagtatttacggtaaactgcccacttggcagtacatcaagtgt |
| | | atcatatgccaagtccgccccctattgacgtcaatgacggtaaatggcccgcctggcattatgccca |
| | | gtacatgaccttacgggactttcctacttggcagtacatctacgtattagtcatcgctattaccatggtg |
| | | atgcggttttggcagtacatcaatgggcgtggatagcggtttgactcacggggatttccaagtctcca |
| | | ccccattgacgtcaatgggagtttgttttggcaccaaaatcaacgggactttccaaaatgtcgtaacaa |
| | | ctccgccccattgacgcaaatgggcggtaggcgtgtacggtgggaggtctatataagcagagctcg |
| | | tttagtgaaccgtcagatcgcctggagacgccatccacgctgttttgacctccatagaagacaccgg |
| | | gaccgatccagcctcccctcgaagccgatctgataacggtaccgataagctggcggccgattaagc |
| | | tacagaagttggtcgtgaggcactgggcaggtaagtatcaaggttacaagacaggtttaaggagac |
| | | caatagaaactgggcttgtcgagacagagaagactcttgcgtttctgataggcacctattggtcttact |
| | | gacatccactttgcctttctctccacaggtgtccactcccaggttcaattacagctcttaagcagccgc |
| | | caaaacaaaattcctcaaaaatcatcatcgaatgaatggtgaaataatttccctgaataactgtagtgtt |
| | | ttcagggcgcggcataataattaactatgctcaaaaattgtgtacctttagctttttaatttgtaaagggg |
| | | ttaataaggaatatttgatgtatagtgccttgactagagatcataatcagccataccacatttgtagagg |
| | | ttttacttgctttaaaaaacctcccacacctccccctgaacctgaaacataaaatgaatgcaattgttgtt |
| | | gttaacttgtttattgcagcttataatggttacaaataaagcaatagcatcacaaatttcacaaataaagc |
| | | atttttttcactgcattctagttgtggtttgtccaaactcatcaatgtatcttatcatgtctggatcatcagat |
| | | ctgccggtctccctatagtgagtcgtattaatttcgataagccaggttaacctgcattaatgaatcggcc |
| | | aacgcgcggggagaggcggtttgcgtattgggcgctcttccgcttcctcgctcactgactcgctgcg |
| | | ctcggtcgttcggctgcggcgagcggtatcagctcactcaaaggcggtaatacggttatccacaga |
| | | atcaggggataacgcaggaaagaacatgtgagcaaaaggccagcaaaaggccaggaaccgtaa |
| | | aaaggccgcgttgctggcgtttttccataggctccgcccccctgacgagcatcacaaaaatcgacg |
| | | ctcaagtcagaggtggcgaaacccgacaggactataaagataccaggcgtttccccctggaagctc |
| | | cctcgtgcgctctcctgttccgaccctgccgcttaccggatacctgtccgcctttctcccttcgggaag |
| | | cgtggcgctttctcatagctcacgctgtaggtatctcagttcggtgtaggtcgttcgctccaagctggg |
| | | ctgtgtgcacgaaccccccgttcagcccgaccgctgcgccttatccggtaactatcgtcttgagtcca |
| | | acccggtaagacacgacttatcgccactggcagcagccactggtaacaggattagcagagcgagg |
| | | tatgtaggcggtgctacagagttcttgaagtggtggcctaactacggctacactagaagaacagtatt |
| | | tggtatctgcgctctgctgaagccagttaccttcggaaaaagagttggtagctcttgatccggcaaac |
| | | aaaccaccgctggtagcggtggtttttttgtttgcaagcagcagattacgcgcagaaaaaaaggatct |
| | | caagaagatcctttgatcttttctacggggtctgacgctcagtggaacgaaaactcacgttaagggatt |
| | | ttggtcatgggcgcgcctaggcttttgcaaagatcgatcaagagacaggatgaggatcgtttcgcag |
| | | cttttcattctgactgcaacgggcaataagtctctgtgtggattaaaaaaagagtgtctgatagcagctt |
| | | ctgaactggttacctgccgtgagtaaattaaaattttattgacttaggtcactaaggcgccttgcgctga |
| | | ggttgcgtcgtgatatcatcagggcagaccggttacatccccctaacaagctgtataaagagaaata |
| | | ctatctcattggcgttgcccgcacctgacagtgcgacgttgggctgcgtccgtcgaccaacggtacc |
| | | gaggtaacagcccaatctatccatgatctcggccaggccgggtcggccgttatgcagcccggctcg |
| | | ggtatgaagccattaaggagccgacccagcgcgaccgggcggccggtcacgctgcctctgctga |
| | | agcctgcctgtcactccctgcgcggcgtacccgccgttctcatcgagtaggctccggatcgcgacc |
| | | ccggacgggccctgggcccaggagcggcctatgacaaatgccgggtagcgatccggcattcagc |
| | | attgactgcgcacggatccagtccttgcaggagccttatgccgaccgtagcaaaaaatgagcccga |
| | | gccgatcgcgagttgtgatccggtcccgccgattgccggtcgcgatgacggtcctgtgtaagcgtta |
| | | tcgttaccaattgtttaagaagtatatacgctacgaggtacttgataacttctgcgtagcatacatgagg |
| | | ttttgtataaaaatggcgggcgatatcaacgcagtgtcagaaatccgaaacagtctgcgggactctg |
| | | gggttcgaaatgaccgaccaagcgacgcccaacctgccatcacgagatttcgattccaccgccgc |
| | | cttctatgaaaggttgggcttcggaatcgttttccgggacgccggctggatgatcctccagcgcggg |
| | | gatctcatgctggagttcttcgcccaccctaggcgcgctcatgagcggatacatatttgaatgtattta |
| | | gaaaaataaacaaataggggttccgcgcacatttccccgaaaagtgccacctaaattgtaagcgtta |
| | | atattttgttaaaattcgcgttaaatttttgttaaatcagctcattttttaaccaataggccgaaatcggcaa |
| | | aatcccttataaatcaaaagaatagaccgagatagggttgagtgttgttccagtttggaacaagagtc |
| | | cactattaaagaacgtggactccaacgtcaaagggcgaaaaaccgtctatcagggcgatggccca |
| | | ctacgtgaaccatcaccctaatcaagttttttggggtcgaggtgccgtaaagcactaaatcggaaccc |
| | | taaagggagcccccgatttagagcttgacggggaaagccggcgaacgtggcgagaaaggaagg |
| | | gaagaaagcgaaaggagcgggcgctagggcgctggcaagtgtagcggtcacgctgcgcgtaac |
| | | caccacacccgccgcgcttaatgcgccgctacagggcgcgtcccattcgccattcaggctgcgca |
| | | actgttgggaagggcgatcggtgcgggcctcttcgctattacgccagctggcgaaagggggatgt |
| | | gctgcaaggcgattaagttgggtaacgccagggttttcccagtcacgacgttgtaaaacgacggcc |
| | | agtgagcgcgcgtaatacgactcactatagggcgaattgggtaccgggccccccctcgaggtcga |
| | | cggtatcgataagcttgatatcgaattcctgcagcccgggggatccactagttctagagcggccgcc |
| | | accgcggtggagctccagcttttgttccctttagtgagggttaattgcgcgcttggcgtaatcatggtc |
| | | atagctgtttcctgtgtgaaattgttatccgctcacaattccacacaacatacgagccggaagcataaa |
| | | gtgtaaagcctggggtgcctaatgagtgagctaactcacattaattgcgttgcgc |

### EXAMPLES

The following non-limiting examples are provided to further illustrate the present invention.

### Example 1. Evaluation of Atlantic Salmon Receiving a DNA Immunomodulator when Formulated with Inactivated IPNV Antigen Using an IPNV Co-Habitation Challenge

The purpose of this study was to determine the efficacy of the DNA immunomodulator administrated to salmon prior to IPNV co-habitation.

### Immunomodulators

The DNA immunomodulator used in this study was a plasmid DNA liposomal complex containing CpG dinucleotides sequences. The plasmid DNA is the pMB75.6 plasmid represented by SEQ ID NO: 2. The plasmid DNA liposomal complex was formulated in a 5% dextrose solution.

The Montanide oil used in this study (Montanide ISA 763 A) is a metabolizable oil that is a type I adjuvant providing a strong and long term immune response. Montanide ISA 763 A VG, supplied by Seppic, contains injectable, metabolizable oils (does not contains mineral oil) and an extremely refined emulsifier obtained from mannitol and purified oleic acid of vegetable origin.

### IPNV Antigen

The IPNV (Infectious pancreas necrosis virus) was propagated in the CHSE-214 cell line until 100% cytopathic effect (CPE) was attained. The antigen was formalin inactivated and then formulated in combination with either one of the immunomodulators listed above at a constant concentration across the various treatments.

### Alpha Ject® 2-2

This commercially available vaccine was used as a control. The vaccine is an oil emulsion used to prevent mortality in fish due to furunculose and IPNV infection.

### IPNV Antigen Vaccine Formulation

The IPNV antigen was formulated as a monovalent vaccine in combination with either the DNA immunomodulator or Montanide oil at three different concentrations/ratios. The treatment composition summary is shown in Table 2.

TABLE 2
Vaccine Formulations.

| | Vaccine formulation | | | | | |
|---|---|---|---|---|---|---|
| | Antigen | Adjuvant | | | Number | |
| | IPNV | DNA | | Montadine | of | Final |
| Treatment | (viral | μg | μg in | ISA 763 | 0.05 ml | volume |
| Group | particles | ml⁻¹ | 0.05 ml | AVG | doses | (ml) |
| A | 0 | 0 | 0 | 0 | 110 | 5.5 |
| | | | | | (90 + 20) | |
| B | 0 | 2 | 0 | 0 | 110 | 5.5 |
| | | | | | (90 + 20) | |
| C | 0 | 20 | 1 | 0 | 110 | 5.5 |
| | | | | | (90 + 20) | |
| D | 0 | 200 | 1 | 0 | 110 | 5.5 |
| | | | | | (90 + 20) | |
| E | 1.5 × 10 | 0 | 0 | 0 | 110 | 5.5 |
| | | | | | (90 + 20) | |
| F | 1.5 × 10 | 2 | 0 | 0 | 120 | 6 |
| | | | | | (90 + 30) | |
| G | 1.5 × 10 | 20 | 1 | 0 | 120 | 6 |
| | | | | | (90 + 30) | |
| H | 1.5 × 10 | 200 | 1 | 0 | 120 | 6 |
| | | | | | (90 + 30) | |
| I | 1.5 × 10 | 0 | 0 | 40:60 | 110 | 5.5 |
| | | | | | (90 + 20) | |
| J | 1.5 × 10 | 0 | 0 | 50:50 | 110 | 5.5 |
| | | | | | (90 + 20) | |
| K | 1.5 × 10 | 0 | 0 | 70:30 | 110 | 5.5 |
| | | | | | (90 + 20) | |

| | | | |
|---|---|---|---|
| L | Alpha Ject ® 2- | 110 | 11 |
| | | (90 + 20) | |

### Study Animals

Atlantic salmon (Salmo salar) were obtained unvaccinated and immune status documented from the supplier. Fish sexually matured, injured or deformed were excluded from the study. The fish were divided into 12 treatment groups (treatment groups A through L) of 30 fish each in three tanks for a total of 360 treatment fish per tank. Another group consisting of 72 fish was used as shedders, or fish actively shedding IPNV, in each of the three tanks. Prior to administration of the vaccine, trial fish were anaesthetized and marked by injection of a PIT-tag two weeks before treatment day.

### Study Design

The fish were starved for a minimum of 48 hours prior to administration of the vaccine. The treatment groups were administered varying doses of the vaccine or control substances described above in Table 2 on the day of treatment. The vaccine or control substances were administered intraperitoneally according to Table 2. The study was performed in three tanks. Each tank had 12 experimental groups of 30 fish each. Two tanks were used as duplicates for the challenge experiment (Tanks 1 and 2, Table 3). The third tank was used for sampling during the immunomodulation study (Tank 3, Table 3).

TABLE 3
Study Design. DNA immunomodulator is abbreviated DNA in this table.

| Group | Vaccine/control substance | No. of fish | Tank and study |
|---|---|---|---|
| A | Dextrose 5% | 30 | Tank 1 - IPNV challenge for |
| B | DNA (2 μg ml⁻¹) | 30 | vaccine testing |
| C | DNA (20 μg ml⁻¹) | 30 | Shedders injected with IPNV with |
| D | DNA (200 μg ml⁻¹) | 30 | a concentration of approx. 10⁷ |
| E | IPNV antigen | 30 | TCID₅₀ ml⁻¹ |
| F | IPNV antigen/DNA (2 μg ml⁻¹) | 30 | |
| G | IPNV antigen/DNA (20 μg ml⁻¹) | 30 | |
| H | IPNV antigen/DNA (200 μg ml⁻¹) | 30 | |
| I | IPNV antigen/Montanide oil (40%) | 30 | |
| J | IPNV antigen/Montanide oil (50%) | 30 | |
| K | IPNV antigen/Montanide oil (70%) | 30 | |
| L | Commercial vaccine as per label | 30 | |
| Shedders | | 72 | |
| A | Dextrose 5% | 30 | Tank 2 - IPNV challenge for |
| B | DNA (2 μg ml⁻¹) | 30 | vaccine testing |
| C | DNA (20 μg ml⁻¹) | 30 | Shedders injected with IPNV with |
| D | DNA (200 μg ml⁻¹) | 30 | a concentration of approx. 10⁷ |
| E | IPNV antigen | 30 | TCID₅₀ ml⁻¹ |
| F | IPNV antigen/DNA (2 μg ml⁻¹) | 30 | |
| G | IPNV antigen/DNA (20 μg ml⁻¹) | 30 | |
| H | IPNV antigen/DNA (200 μg ml⁻¹) | 30 | |
| I | IPNV antigen/Montanide oil (40%) | 30 | |
| J | IPNV antigen/Montanide oil (50%) | 30 | |
| K | IPNV antigen/Montanide oil (70%) | 30 | |
| L | Commercial vaccine as per label | 30 | |
| Shedders | | 72 | |
| A | Dextrose 5% | 30 | Tank 3 - Immunomodulation |
| B | DNA (2 μg ml⁻¹) | 30 | study, Sampling tank |
| C | DNA (20 μg ml⁻¹) | 30 | Shedders injected with IPNV with |
| D | DNA (200 μg ml⁻¹) | 30 | a concentration of approx. 10⁷ |
| E | IPNV antigen | 30 | TCID₅₀ ml⁻¹ |
| F | IPNV antigen/DNA (2 μg ml⁻¹) | 30 | |
| G | IPNV antigen/DNA (20 μg ml⁻¹) | 30 | |
| H | IPNV antigen/DNA (200 μg ml⁻¹) | 30 | |
| I | IPNV antigen/Montanide oil (40%) | 30 | |
| J | IPNV antigen/Montanide oil (50%) | 30 | |
| K | IPNV antigen/Montanide oil (70%) | 30 | |
| L | Commercial vaccine as per label | 30 | |

| | |
|---|---|
| Shedders | 48 |

The challenge experiments were performed in connection with transfer to sea water after a minimum of 600 degree days, approximately 6 weeks post vaccination and photoperiod manipulation. The fish were starved for a minimum of 25 hours prior to challenge. All tanks were challenged at the same time. The shedders were intraperitoneally injected with the cultivated virus and marked by cutting the adipose fin at time of challenge. Following injection and marking, the shedders were introduced. Fish originally from Tanks 1 and 2 were allocated to tanks M5 and M6, and fish originally from Tank 3 were transferred to tank M2.

### Sampling

For each time point, fish were culled and tissue, e.g. head-kidney, liver and spleen, samples obtained for immunoprofiling. Blood was also collected for serology and/or transcriptome analysis. At each time point, 5 fish per treatment were culled and sampled. Before vaccination, 10 fish from the general population were sampled. After vaccination, samples from tank 3 were collected at 24 and 48 hours post-vaccination. After challenge, samples from tanks 1 and 2 were taken for standard confirmatory loss (10% of mortality). Samples from tank 3 were collected at 3 and 7 days post-challenge. At the end of the challenge period, the surviving fish were culled and sampled.

A representative selection of dead fish from the IPNV challenge tanks (tanks 1 and 2) were kidney sampled and tested in an IPNV Ag ELISA. The selection was done in the start, middle, and the end of the mortality up to a total of 10% of the fish (30/tank).

A representative selection of the dead fish during challenge (˜10% of the total number of fish included in the trial) were examined bacteriologically.

### Results

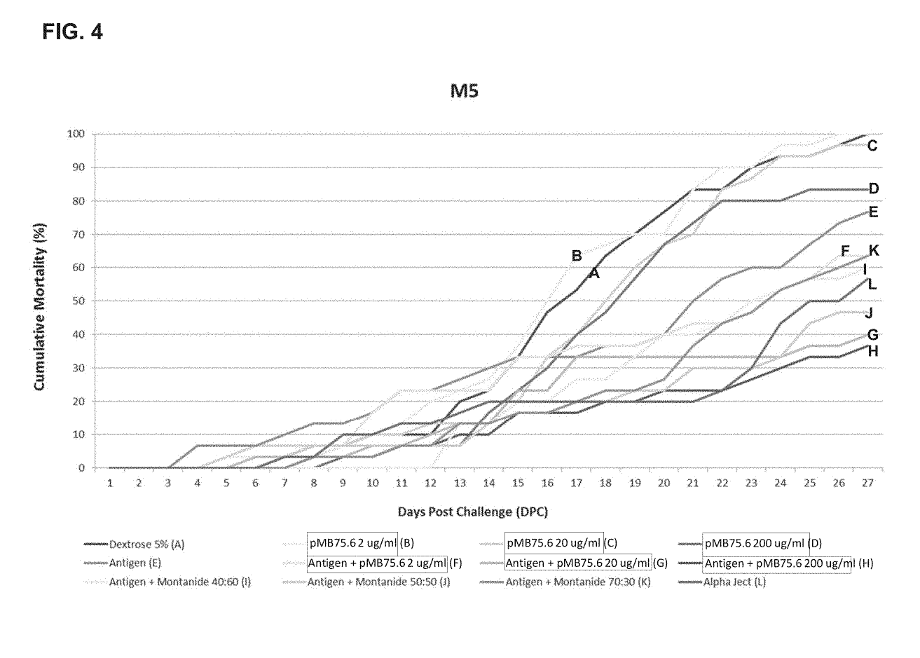

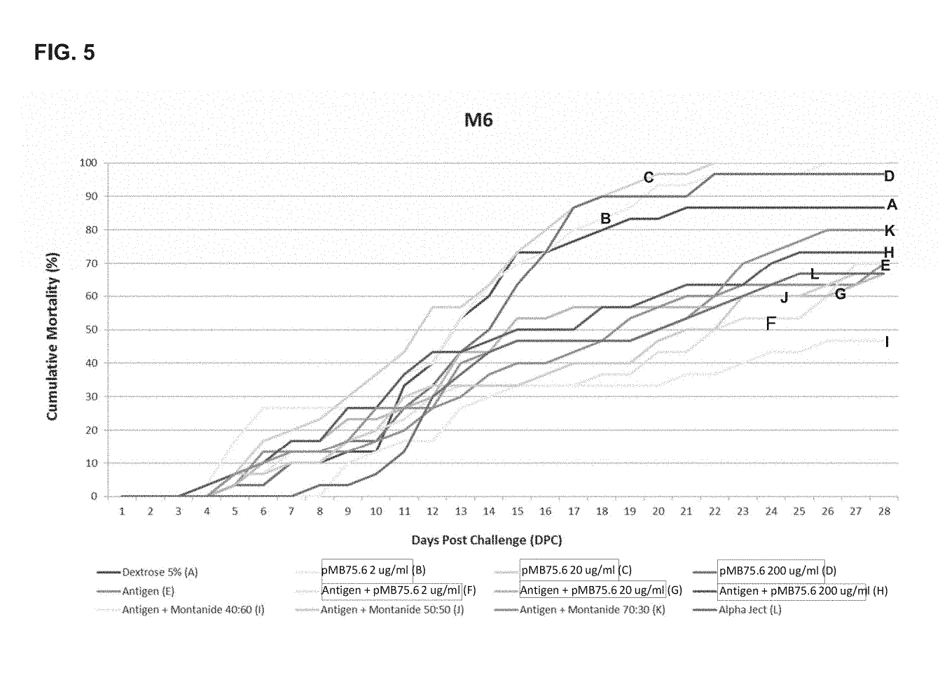

Following the introduction of the shedders, the challenge study was allowed to progress until mortality leveled off or reached 100% in the negative control groups. Plateaus in the mortality were reached between day 22 and 28 days post-infection in tanks 1 and 2 (FIG. 4 & FIG. 5).

Deceased fish were sampled using an IPNV Ag ELISA and bacteriological analysis. IPNV Ag ELISA testing revealed 75.5% of fish from tank M6 being positive for IPNV, 62.5% of fish in tank M5 and just 35.2% of fish in tank M2 (Table 4). Samples taken from 10% of the mortality were submitted for bacteriology analysis. The percentage of samples with no bacterial growth taken from fish in the various tanks were 77.5%, 64.9% and 98.1% for tanks M5, M6 and M2 respectively (Table 5). The majority of mortality recorded subsequently were negative for bacterial growth.

TABLE 4
IPNV Ag ELISA results.

| | Total No. fish | Total No. |
|---|---|---|
| Tank | [symbol] | [symbol] |
| M5 | 40 (26.5%) | 25 (62.5%) |
| M6 | 57 (37.7%) | 43 (75.5%) |
| M2 | 54 (35.8%) | 19 (35.2%) |

[symbol]

indicates data missing or illegible when filed

TABLE 5
Bacteriological results from tanks M5, M6 and M2.

| | | Total No. | Total No. | Total No. |
|---|---|---|---|---|
| | Date of | fish | bacteriology | bacteriology |
| Tank | sampling | tested | samples negative | samples positive |
| M5 | 01/07/2014 | 3 | 3 | — |
| | 02/07/2014 | 5 | 4 | 1 |
| | 03/07/2014 | 3 | 3 | — |
| | 04/07/2014 | 1 | 1 | — |
| | 05/07/2014 | 4 | 4 | — |
| | 08/07/2014 | 7 | — | 7 |
| | 09/07/2014 | 5 | 5 | — |
| | 18/07/2014 | 11 | 11 | — |
| | 21/07/2014 | 1 | 0 | 1 |
| | Total | 40 | 31 (77.5%) | 9 (22.5%) |
| M6 | 01/07/2014 | 12 | 7 | 5 |
| | 08/07/2014 | 12 | — | 12 |
| | 13/07/2014 | 8 | 7 | 1 |
| | 14/07/2014 | 6 | 4 | 2 |
| | 15/07/2014 | 4 | 4 | |
| | 16/07/2014 | 8 | 8 | — |
| | 17/07/2014 | 5 | 5 | — |
| | 18/07/2014 | 2 | 2 | — |
| | Total | 57 | 37 (64.9%) | 20 (35.1%) |
| M2 | 03/07/2014 | 2 | 2 | — |
| | 04/07/2014 | 1 | 1 | — |
| | 05/07/2014 | 1 | 1 | — |
| | 10/07/2014 | 4 | 4 | — |
| | 15/07/2014 | 4 | 4 | — |
| | 23/07/2014 | 42 | 41 | 1 |
| | Total | 54 | 53 (98.1%) | 1 (1.9%) |

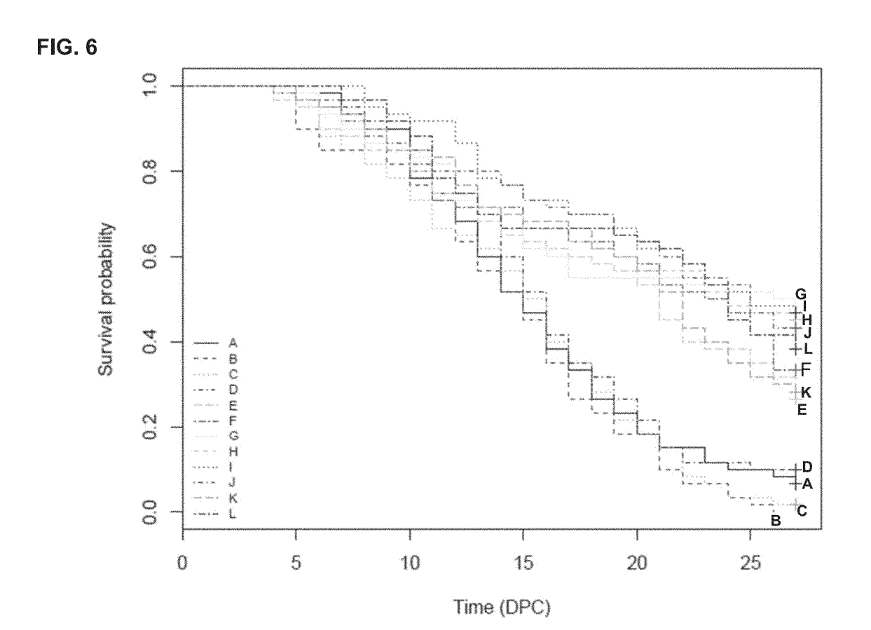

Kaplan-Meier survival curves of the combined data were determined in order to understand and compare the survival times from each treatment group (group A to L). As observed in FIG. 6, the survival probability was secluded in two distinct populations. The population with lower survival probability was composed of fish treated with dextrose (A) or the 3 different doses of the DNA immunomodulator without antigen (B, C and D) while the population showing a greater survival probability was composed of the groups treated with only IPNV antigen (E), the IPNV antigen formulated with the DNA immunomodulator (F, G and H) or with Montanide oil (I, J and K), and the commercial vaccine (L). When compared as a whole, statistically significant differences were found in the survival curves between the 12 different treatments (Chi-square=110.6 df=11; p-values (<0.0001)=2.2×10−16).

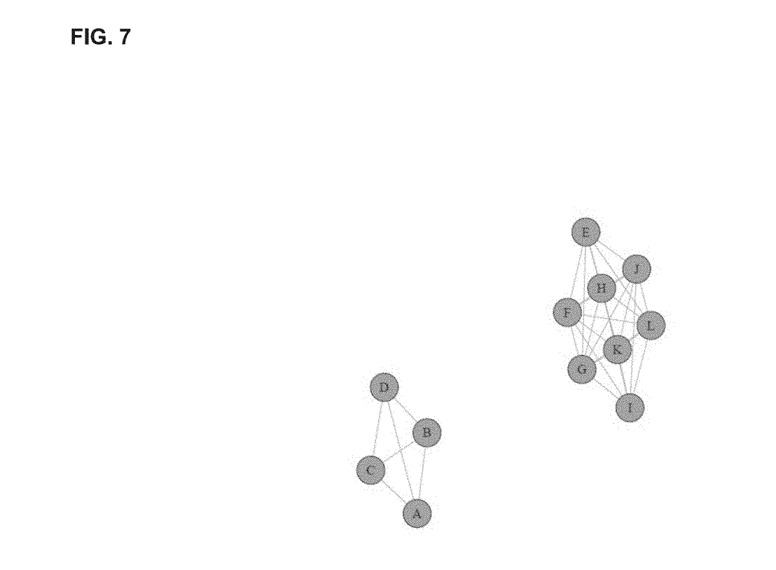

The treatments were divided among 2 distinct clusters (FIG. 7). One cluster included the dextrose-treatment (A) as well as all the DNA immunomodulator without antigen treatments (B, C, D). The other cluster was composed of fish treated with IPNV antigen only (E), the DNA immunomodulator formulated vaccines (F, G, H), the Montanide formulated antigen (I, J, K), and the commercial vaccine (L).

Significant difference between the two clusters was evident thus confirming that fish survival was significantly improved by vaccination compared to the negative control groups however no distinction was seen between the different vaccine formulations.

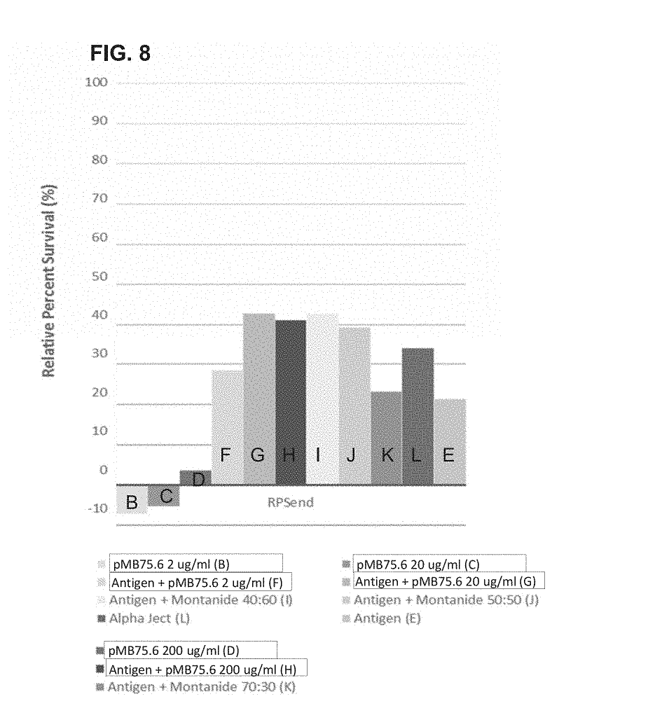

Relative percent survival was calculated in all treatment groups at the end (RPSend) of the experiment (FIG. 8). Significant differences were measured between 5% dextrose negative control group and all the formulated vaccines.

The Speilberg scale was used to access possible negative effects of the adjuvants used to formulate the vaccines. No particular secondary effects were observed. Tissue lesions were almost non-existent with only occasional level 2 (low) score for adhesions and pigmentation in the commercial product and level 1 scoring for the DNA immunomodulator (Table 6). No vaccine residues were detectable in any of the fish sampled.

TABLE 6
Spielberg scoring for tanks M5 and M6.

| | | | Visceral | | Vaccine |
|---|---|---|---|---|---|
| Tank | Gro | No. Fish | adhesions | Pigmentation | residues |
| M5 | E | 5 | 0 | 0 | 0 |
| | F | 7 | 0 | 0 | 0 |
| | G | 17 | 0 | 0 | 0 |
| | H | 16 | 1 in 2 | 1 in 1 fish | 0 |
| | I | 12 | 1 in 1 | 1 in 1 fish | 0 |
| | J | 14 | 0 | 0 | 0 |
| | K | 9 | 1 in 2 | 1 in 1 fish | 0 |
| | L | 9 | 1 in 4 | 1 in 7 fish | 0 |
| | | | fish | 2 in 2 fish | |
| M6 | A | 1 | 0 | 3 in 1 fish | 0 |
| | E | 5 | 0 | 0 | 0 |
| | F | 7 | 0 | 0 | 0 |
| | G | 8 | 0 | 0 | 0 |
| | H | 5 | 1 in 1 | 0 | 0 |
| | I | 13 | 1 in 1 | 0 | 0 |
| | J | 8 | 0 | 1 in 1 fish | 0 |
| | K | 5 | 0 | 0 | 0 |
| | L | 10 | 1 in 7 | 1 in 5 fish | 0 |
| | | | fish | 2 in 1 fish | |
| Combined | A | 1 | 0 | 3 in 1 fish | 0 |
| data | B | 1 | 0 | 0 | 0 |
| | D | 1 | 0 | 0 | 0 |
| | E | 14 | 0 | 0 | 0 |
| | F | 21 | 0 | 0 | 0 |
| | G | 32 | 0 | 0 | 0 |
| | H | 19 | 1 in 3 | 1 in 1 fish | 0 |
| | I | 25 | 1 in 2 | 1 in 1 fish | 0 |
| | J | 22 | 0 | 1 in 1 fish | 0 |
| | K | 14 | 1 in 2 | 1 in 1 fish | 0 |
| | L | 19 | 1 in 11 | 1 in 12 fish | 0 |
| | | | fish | 2 in 3 fish | |
| | | | 2 in 8 | | |
| | | | fish | | |

### CONCLUSION

In this study, the efficacy of a DNA immunomodulator formulated with inactive IPNV as a vaccine in Atlantic salmon was assessed. When the DNA immunomodulator was administered alone (treatments B, C and D) it gave minimal protection, with mortality ranging between 93.3 to 100% and survival probabilities not significantly different than the dextrose-only control group. This was expected as the innate immune response elicited by the DNA immunomodulator would have most likely dissipated by the challenge time point (588 degree days or approximately 7 weeks post-vaccination) although this was not assessed in the current study.

As depicted in FIG. 8, the presence of the IPNV inactivated antigen alone was sufficient in significantly increasing the survival probability of vaccinated fish with a RPSend of 21.4% compared to the 5% dextrose group. The protection was further increased with the adjuvanted formulations of the DNA immunomodulator (F=28.7%, G=42.9%, H=41.1%) and Montanide (1=42.9%, J=39.3%, K=23.2%). On the safety aspect, no issues were recorded and the DNA immunomodulator was deemed safe for use in fish.

This study showed the potential use of the DNA immunomodulator as a fish vaccine adjuvant, its ability to help induce an immune response in Atlantic salmon as well as its safety profile when administered at 10 ug or lower.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above products, compositions, and methods without departing from the scope of the invention, it is intended that all matter contained in the above description and shown in the accompanying drawings shall be interpreted as illustrative and not in a limiting sense.

What is claimed is:

1. A method of eliciting an immune response in a recipient aquatic species subject comprising:
a. administering an effective amount of an immunomodulator composition to an aquatic species subject, wherein the immunomodulator composition comprises a nucleic acid sequence comprising at least one immunostimulatory CpG motif, at least one non-immunostimulatory CpG motif, and a cationic liposome.

2. The method of claim 1, wherein the liposome delivery vehicle comprises lipids selected from the group consisting of multilamellar vesicle lipids and extruded lipids.

3. The method of claim 1, wherein the liposome delivery vehicle comprises pairs of lipids selected from the group consisting of N-[1-(2,3-dioleyloxy)propyl]-N,N,N-trimethylammonium chloride (DOTMA) and cholesterol; N-[1-(2,3-dioleoyloxy)propyl]-N,N,N-trimethylammonium chloride (DOTAP) and cholesterol; 1-[2-(oleoyloxy)ethyl]-2-oleyl-3-(2-hydroxyethyl)imidazolinium chloride (DOTIM) and cholesterol; and dimethyldioctadecylammonium bromide (DDAB) and cholesterol.

4. The method of claim 1, wherein the immunomodulator composition further comprises a biological agent.

5. The method of claim 1, wherein the administration is before exposure to an infectious agent.

6. The method of claim 1, wherein the administration is after exposure to an infectious agent.

7. The method of claim 1, wherein the immunomodulator composition comprises a nucleic acid sequence having at least 80% sequence identity with a sequence selected from the group consisting of SEQ ID NO: 1, SEQ ID NO: 2, SEQ ID NO: 3, and SEQ ID NO: 4.

8. The method of claim 4, wherein the biological agent is selected from the group consisting of immune enhancer proteins, immunogens, vaccines, antimicrobials, and any combination thereof.

9. The method of claim 1, wherein the aquatic species subject is a fish.

10. The method of claim 9, wherein the fish is a salmon.

11. The method of claim 9, wherein the fish is a catfish.

12. The method of claim 9, wherein the fish is a carp.

13. The method of claim 9, wherein the fish is a trout.

14. The method of claim 9, wherein the fish is a yellow tail.

15. The method of claim 9, wherein the fish is a bass.

16. The method of claim 9, wherein the subject is an aqua culture raised fish.

17. The method of claim 1 further comprising a pharmaceutically acceptable carrier.

18. A method of improving production of aqua culture raised fish comprising administering to the fish an immunomodulator composition, wherein the immunomodulator composition comprises a nucleic acid sequence comprising at least one immunostimulatory CpG motif, and a cationic liposome.

19. A method of improving survivability of aqua culture raised fish comprising administering to the fish an immunomodulator composition, wherein the immunomodulator composition comprises a nucleic acid sequence comprising at least one immunostimulatory CpG motif, and a cationic liposome.

20. A method of improving an immune response to a vaccine antigen comprising administering to a recipient aquatic species subject an immunomodulator composition, wherein the immunomodulator composition comprises a nucleic acid sequence comprising at least one immunostimulatory CpG motif, and a cationic liposome.
